SEQUENCE LISTING (45 sequences)

SEQ ID NO: 1 - protein, 6 aa, Homo sapiens
LPPHSS

SEQ ID NO: 2 - protein, 7 aa, Homo sapiens
SLPPHSS

SEQ ID NO: 3 - protein, 7 aa, Homo sapiens
LPPHSSQ

SEQ ID NO: 4 - protein, 8 aa, Homo sapiens
SLPPHSSQ

SEQ ID NO: 5 - protein, 8 aa, Homo sapiens
TSLPPHSS

SEQ ID NO: 6 - protein, 8 aa, Homo sapiens
LPPHSSQS

SEQ ID NO: 7 - protein, 9 aa, Homo sapiens
TSLPPHSSQ

SEQ ID NO: 8 - protein, 9 aa, Homo sapiens
SLPPHSSQS

SEQ ID NO: 9 - protein, 10 aa, Homo sapiens
TSLPPHSSQS

SEQ ID NO: 10 - protein, 9 aa, Homo sapiens
ATSLPPHSS

SEQ ID NO: 11 - protein, 10 aa, Homo sapiens
ATSLPPHSSQ

SEQ ID NO: 12 - protein, 11 aa, Homo sapiens
ATSLPPHSSQS

SEQ ID NO: 13 - protein, 9 aa, Homo sapiens
  MISC_FEATURE (1)..(1): May be biotinylated
LPPHSSQSP

SEQ ID NO: 14 - protein, 10 aa, Homo sapiens
SLPPHSSQSP

SEQ ID NO: 15 - protein, 11 aa, Homo sapiens
TSLPPHSSQSP

SEQ ID NO: 16 - protein, 12 aa, Homo sapiens
ATSLPPHSSQSP

SEQ ID NO: 17 - protein, 12 aa, Homo sapiens
ATSLPPHSSLQT

SEQ ID NO: 18 - protein, 13 aa, Homo sapiens
ATSLPPHSSQSPL

SEQ ID NO: 19 - protein, 15 aa, Homo sapiens
ATSLPPHSSQSPRAL

SEQ ID NO: 20 - protein, 8 aa, Homo sapiens
SLPPRALQ

SEQ ID NO: 21 - protein, 8 aa, Homo sapiens
TSLPPRAL

SEQ ID NO: 22 - protein, 8 aa, Homo sapiens
LPPRALQS

SEQ ID NO: 23 - protein, 9 aa, Homo sapiens
TSLPPRALQ

SEQ ID NO: 24 - protein, 9 aa, Homo sapiens
SLPPRALQS

SEQ ID NO: 25 - protein, 10 aa, Homo sapiens
TSLPPRALQS

SEQ ID NO: 26 - protein, 9 aa, Homo sapiens
ATSLPPRAL

SEQ ID NO: 27 - protein, 10 aa, Homo sapiens
ATSLPPRALQ

SEQ ID NO: 28 - protein, 11 aa, Homo sapiens
ATSLPPRALQS

SEQ ID NO: 29 - protein, 9 aa, Homo sapiens
LPPRALQSP

SEQ ID NO: 30 - protein, 10 aa, Homo sapiens
SLPPRALQSP

SEQ ID NO: 31 - protein, 11 aa, Homo sapiens
TSLPPRALQSP

SEQ ID NO: 32 - protein, 12 aa, Homo sapiens
ATSLPPRALQSP

SEQ ID NO: 33 - protein, 7 aa, Homo sapiens
WLPPHSS

SEQ ID NO: 34 - protein, 12 aa, Homo sapiens
ATWLPPHSSQSP

SEQ ID NO: 35 - protein, 7 aa, Homo sapiens
WLPPRAL

SEQ ID NO: 36 - protein, 12 aa, Homo sapiens
ATWLPPRALQSP

SEQ ID NO: 37 - protein, 12 aa, Homo sapiens
PSQSSHPPLSTA

SEQ ID NO: 38 - protein, 7 aa, Homo sapiens
SLPAHAR

SEQ ID NO: 39 - protein, 7 aa, Homo sapiens
HSSLQTP

SEQ ID NO: 40 - protein, 7 aa, Homo sapiens
YHSSFQA

SEQ ID NO: 41 - protein, 15 aa, Homo sapiens
ATSLPPHECQSPLRC

SEQ ID NO: 42 - protein, 25 aa, Homo sapiens
ALAQSHVLPPHSSQSPPLCARPPHA

SEQ ID NO: 43 - protein, 7 aa, Homo sapiens
ATWLPPR

SEQ ID NO: 44 - protein, 7 aa, Homo sapiens
TPHNTVS

SEQ ID NO: 45 - protein, 19 aa, Homo sapiens
SPWSSCSVTCGDGVITRIR

(19) United States
(12) Patent Application Publication
(10) Pub. No.: US 2009/0047335 A1
(43) Pub. Date: Feb. 19, 2009

(54) Anti-angiogenic peptides and methods of use thereof

(75) Inventors: Luca Rastelli, New Haven, CT (US); Mary Kay Lescoe, New Haven, CT (US); Melissa Corso, New Haven, CT (US); Richard Kitson, New Haven, CT (US); Judith Landin, New Haven, CT (US); Lina Souan, New Haven, CT (US); Uriel M. Malyankar, New Haven, CT (US)

Correspondence Address: COOLEY GODWARD KRONISH LLP;ATTN: Patent Group, Suite 1100, 777 - 6th Street, NW, WASHINGTON, DC, 20001, US

(73) Assignee: SOPHERION THERAPEUTICS, INC., NEW HAVEN, CONNECTICUT, CT (US)

(21) Appl. No.: 11/659,731

(22) PCT Filed: Aug. 5, 2005
(86) PCT No.: PCT/US05/27883

§ 371 Date: Sep. 26, 2008

Related U.S. Application Data
(60) Provisional application No. 60/599,059, filed on Aug. 6, 2004.

(51) Int. Cl. A61K 9/127; C07K 7/00; A61K 9/14; C12N 1/20; A61P 31/00; C40B 50/00; C12N 5/06; A61K 38/00

(52) U.S. Cl. 424/450; 530/327; 530/328; 530/329; 514/17; 514/16; 514/15; 514/14; 424/501; 435/375; 435/252.1; 506/23

(57) ABSTRACT

Anti-angiogenic peptides that inhibit VEGF-mediated activation or proliferation of endothelial cells are disclosed. Such peptides may be used to inhibit VEGF binding to the VEGFR2 receptor (also known as the kinase domain receptor or KDR). Such peptides may also be used to inhibit VEGF-mediated activation of endothelial cells in angiogenesis-associated diseases such as cancer, inflammatory diseases, eye diseases and skin disorders.

### FIELD OF INVENTION

This application relates to the identification and design of therapeutic peptides for treatment and characterization of angiogenesis-related diseases, particularly anti-angiogenic peptides that block binding of vascular endothelial growth factor (VEGF) to its receptor, VEGFR2, also known as the kinase domain receptor or kinase insert domain-containing receptor (KDR).

### BACKGROUND OF INVENTION

Angiogenesis is the process by which new blood vessels form by developing from pre-existing vessels. This multi-step process involves signaling to endothelial cells, which results in (1) dissolution of the membrane of the originating vessel, (2) migration and proliferation of the endothelial cells, and (3) formation of a new vascular tube by the migrating cells (Alberts et al., 1994, Molecular Biology of the Cell. Garland Publishing, Inc., New York, N.Y. 1294 pp.). While this process is employed by the body in beneficial physiological events such as wound healing and myocardial infarction repair, it is also exploited by unwanted cells such as tumor cells, and in undesirable conditions such as atherosclerosis, inflammatory conditions such as dermatitis, psoriasis, and rheumatoid arthritis, as well as eye diseases such as diabetic retinopathy and macular degeneration.

Angiogenesis is required for the growth and metastasis of solid tumors. Studies have confirmed that in the absence of angiogenesis, tumors rarely have the ability to develop beyond a few millimeters in diameter (Isayeva et al., 2004, Int. J. Oncol. 25(2):335-43). Angiogenesis is also necessary for metastasis formation by facilitating the entry of tumor cells into the blood circulation and providing new blood vessels that supply nutrients and oxygen for tumor growth at the metastatic site (Takeda et al., 2002, Ann Surg. Oncol. 9(7):610-16).

Endothelial cells are also active participants in chronic inflammatory diseases, in which they express various cytokines, cytokine receptors and proteases that are involved in angiogenesis, proliferation and tissue degradation. For example, during rheumatoid arthritis, endothelial cells become activated and express adhesion molecules and chemokines, leading to leukocyte migration from the blood into the tissue. Endothelial cell permeability increases, leading to edema formation and swelling of the joints (Middleton et al., 2004, Arthritis Res. Ther. 6(2):60-72).

Abnormal neovascularization is also seen in various eye diseases, where it results in hemorrhage and functional disorder of the eye, contributing to the loss of vision associated with such diseases as retinopathy of prematurity, diabetic retinopathy, retinal vein occlusion, and age-related macular degeneration (Yoshida et al., 1999, Histol Histopathol. 14(4): 1287-94). These conditions are the leading causes of blindness among infants, those of working age and the elderly (Aiello, 1997, Ophthalmic Res. 29(5):354-62).

Understanding angiogenesis is also of crucial importance for the treatment of skin diseases, as it is a key contributor to pathologic dermatological processes such as psoriasis, warts, cutaneous malignancy, decubitus ulcers, stasis ulcers, pyogenic granulomas, hemangiomas, Kaposi's sarcoma, and possibly Spitz nevus, hypertrophic scars, and keloids (Arbiser, 1996, J. Am. Acad. Dermatol. 34(3):486-97). Thus, recent developments in the understanding of angiogenesis will likely lead to advances in the treatment of skin cancer, psoriasis and other skin diseases, and more rapid healing of wounds.

Vascular endothelial growth factor (VEGF) is a particularly potent angiogenic factor that acts as an endothelial cell-specific mitogen during angiogenesis (Binetruy-Tourniere et al., 2000, EMBO J. 19(7): 1525-33). VEGF has been implicated in promoting solid tumor growth and metastasis by stimulating tumor-associated angiogenesis (Lu et al., 2003, J. Biol. Chem. 278(44): 43496-43507). VEGF has been found in the synovial fluid and serum of patients with rheumatoid arthritis (RA), and its expression is correlated with disease severity (Clavel et al., Joint-Bone-Spine. 2003 70(5):321-6). VEGF has also been implicated as a major mediator of intraocular neovascularization and permeability. Transgenic mice overexpressing VEGF demonstrate clinical intraretinal and subretinal neovascularization, and form leaky intraocular blood vessels detectable by angiography, demonstrating their similarity to human disease (Miller, 1997, Am. J. Pathol. 151(1):13-23).

Given the involvement of pathogenic angiogenesis in such a wide variety of disorders and diseases, inhibition of angiogenesis, and particularly of VEGF signaling, is a desirable therapeutic goal. VEGF acts through two high affinity tyrosine kinase receptors, VEGFR1 (or fms-like tyrosine kinase, Flt-1), and VEGFR (also known as kinase domain receptor or kinase insert domain-containing receptor, KDR). Although VEGFR1 binds VEGF with a 50-fold higher affinity than KDR, KDR appears to be the major transducer of VEGF angiogenic effects, i.e., mitogenicity, chemotaxis and induction of tube formation (Binetruy-Toumiere et al., supra). Inhibition of KDR-mediated signal transduction by VEGF, therefore, represents an excellent approach for anti-angiogenic intervention.

In this regard, inhibition of angiogenesis and tumor inhibition has been achieved by using agents that either interrupt VEGF/KDR interaction and/or block the KDR signal transduction pathway including: antibodies to VEGF (Kim et al., 1993, Nature 362, 841-844; Kanai et al., 1998, J. Cancer 77, 933-936; Margolin et al., 2001, J. Clin. Oncol. 19, 851-856); antibodies to KDR (Lu et al., 2003, supra; Zhu et al., 1998, Cancer Res. 58, 3209-3214; Zhu et al. 2003, Leukemia 17, 604-611; Prewett et al., 1999, Cancer Res. 59, 5209-5218); anti-VEGF immunotoxins (Olson et al., 1997, Int. J. Cancer 73, 865-870); ribozymes (Pavco et al., 2000, Clin. Cancer Res. 6, 2094-203); soluble receptors (Holash et al, 2002, Proc. Natl. Acad. Sci. USA 99, 11393-11398; Clavel et al. supra); tyrosine kinase inhibitors (Fong et al., 1999, Cancer Res. 59, 99-106; Wood et al, 2000, Cancer Res. 60, 2178-2189; Grosios et al., 2004, Inflamm Res. 53(4):133-42); antisense mediated VEGF suppression (Forster et al, 2004, Cancer Lett. 20; 212(1):95-103); and RNA interference (Takei et al., 2004, Cancer Res. 64(10):3365-70; Reich et al., 2003, Mol. Vis. 9:210-6). Peptides that block binding of VEGF to KDR have also been described, and were shown to inhibit VEGF-induced angiogenesis in a rabbit corneal model (Binetruy-Toumiere et al, 2000, EMBO J. 19(7): 1525-33). Still, given the wide variety of patients that stand to benefit from the development of effective anti-angiogenic treatments, there remains a need for the further identification and characterization of novel anti-angiogenic drug compounds.

Recently, Genentech introduced to the market a recombinant humanized anti-VEGF monoclonal antibody, Avastin (bevacizumab). This antibody has shown efficacy in the treatment of colon cancer, and is being tested on other tumor cell types. Cost analysis suggests that treatment with this antibody could add from $42,800 to $55,000 per patient to the cost of care for advanced colorectal cancer, or more than $1.5 billion annually in the United States. Thus, there is a need for alternative drugs such as small peptides that are less expensive to manufacture and may be used therapeutically at a much lower cost.

### SUMMARY OF INVENTION

The present inventors have identified using mini peptide display technology novel anti-angiogenic peptides that block or reduce VEGF-induced stimulation of endothelial cell activation or proliferation. The peptides of the invention provide an improvement over the prior art, in that at least some of the inventive peptides demonstrate a significantly lower IC₅₀ when compared to previously known peptides. Accordingly, the peptides of the invention are useful for the treatment of angiogenesis-related diseases, including the treatment of tumors and neoplasias, inflammatory diseases such as rheumatoid arthritis and psoriasis, vascular disorders including atherosclerosis, vascular restenosis, arteriovenous malformations and vascular adhesion pathologies, and eye diseases including diabetic retinopathy and macular degeneration.

### BRIEF DESCRIPTION OF THE DRAWINGS

FIG. 1 shows a phylogenetic tree generated by custalW multiple sequence alignment algorithm using Vector NTI, which compares the relationship between the peptides identified using mini peptide display technology and the peptides disclosed in Binetruy-Toumaire R, Demangel C, Malavaud B, Vassy R, Rouyre S, Kraemer M, Plouet J, Derbin C, Perret G, Mazie J C. EMBO J. 2000 Apr. 3; 19(7):1525-33, and Lu D, Shen J, Vil M D, Zhang H, Jimenez X, Bohlen P, Witte L, Zhu Z. J Biol. Chem. 2003 October 31; 278(44):43496-507.

FIG. 2 shows a homology alignment between the following peptides: EmboK4 (SEQ ID No. 38), EmboK5 (SEQ ID No. 39) and EmboV4 (SEQ ID No. 40) from the paper by Binetruy-Toumaire et al., the two peptides 1A11 and 2D5 (which have the same sequence (SEQ ID No. 41) and therefore will be considered as one) from the paper by Lu et al., and the clone K3 (SEQ ID No. 42) obtained by mini peptide display technology.

FIG. 3 shows a further homology alignment including K3 and the two of the peptides disclosed by Binetruy-Toumaire et al., EmboV1 (SEQ ID No. 43) and EmboK3 (SEQ ID No. 44).

FIG. 4 is a graph showing VEGF-mediated survival/proliferation of bovine retinal endothelial cells (BRE cells) in the presence of various peptides.

FIG. 5 shows micrographs of the number and morphology BRE cells exposed to various treatments, including (A) no VEGF, (B) VEGF, (C) VEGF plus 100 microgram/ml of ST100,038, (D) VEGF plus 50 microgram/ml of ST100,038, and (E) VEGF plus 100 microgram/ml of ST100,039.

FIG. 6 is a graph showing VEGF-mediated survival/proliferation of BRE cells in the presence of various peptides, where the maximum concentration of peptide was increased to 200 microgram/ml.

FIG. 7 is a graph showing VEGF-mediated survival/proliferation of BRE cells in the presence of ST100,038 peptide containing L amino acids versus the same sequence containing D amino acids (peptide ST100,045).

FIG. 8 shows micrographs of the number and morphology of BRE cells exposed to various treatments, including (A) no VEGF, (B) VEGF, (C) VEGF plus 50 microgram/ml of ST100,038, (D) VEGF plus 50 microgram/ml of ST100,045.

FIG. 9 is a graph showing VEGF-mediated survival/proliferation of BRE cells in the presence of peptides ST100,038 and ST100,045 after the cells had adhered.

FIG. 10 is a graph showing VEGF-mediated survival/proliferation of BRE cells in the presence of peptides ST-100,038 and ST100,045 before the cells had adhered.

FIG. 11 is a graph showing VEGF-mediated survival/proliferation of BRE cells in the presence of peptide ST100,038 and 10% fetal bovine serum.

FIG. 12 is a graph showing VEGF-mediated survival/proliferation of BRE cells in the presence of peptides ST100,038 and ST100,045 after the cells had adhered.

FIG. 13 is a graph showing the effects of the peptide ST100,038 and the retro inverso peptide ST100,059 on VEGF-mediated HUVEC survival.

FIG. 14 shows photographs of angioreactors after removal from mice treated with various peptides and represent a qualitative appreciation of the level of angiogenesis. Photograph (A) shows the angioreactors from mice treated with VEGF plus 160 μM peptide ST100,038. Photograph (B) shows the angioreactors from mice treated with VEGF plus 40 μM peptide ST100,038. Photograph (C) shows the angioreactors from mice treated with VEGF plus 160 μM peptide ST100,045. Photograph (D) shows the angioreactors from mice treated with VEGF plus 40 μM peptide ST100,045. Photograph (E) shows the angioreactors from mice treated with PBS alone. Photograph (F) shows the angioreactors from mice treated with VEGF alone. Photograph (G) shows the angioreactors from mice treated with VEGF plus peptide TSP616.

FIG. 15 is a bar graph comparing inhibition of VEGF-mediated angiogenesis in vivo with ST100,038 and ST100,045 as compared to unstimulated PBS and TSP616 controls.

FIG. 16 is a graph comparing inhibition of VEGF-mediated angiogenesis in vivo with ST100,038 and ST100,059 as compared to unstimulated PBS and TSP616 controls. In addition, below each group, there are photographs of angioreactors after removal from mice treated with various peptides and represent a qualitative appreciation of the level of angiogenesis.

FIG. 17 is a graph comparing inhibition of the growth of a subcutaneous B16 melanoma tumor in C57BL/6 mice treated with 20 mg/kg, 40 mg/kg, 100 mg/kg daily ip of ST100,059 to untreated controls.

FIG. 18 is a graph comparing the number of B16 melanoma lung metastases in mice treated with ST100,059 administered ip either 100 mg/kg daily or 100 mg/kg every 2 days to untreated controls.

FIG. 19 is a graph comparing caliper measurements and actual tumor weights of the human breast cancer tumor line MDA-MB231 in mice treated with vehicle, docetaxel, 10 mg/kg daily or 20 mg/kg daily of ST100,059.

FIG. 20 is a graph comparing the number of animals with tumor necrosis and the extent of necrosis for each MDA-MB231 human breast cancer tumor in mice treated with vehicle, 10 mg/kg daily or 20 mg/kg daily of ST100,059.

FIG. 21 is a graph showing that increasing concentrations of the peptide ST100,059 reduce the level of protein kinase MPK phosphorylation in HUVEC.

FIG. 22 is an image showing that the peptide ST100,059 regulates VEGF induced gene expression changes, using the Down Syndrome critical region gene 1 as an example.

FIG. 23 is an image showing that the peptide ST100,059 regulates VEGF induced gene expression changes, using the peptidyl arginine deiminase type 1 gene as an example.

### DETAILED DESCRIPTION OF THE INVENTION

### Peptides

The present inventors have identified novel anti-angiogenic peptides. The term “anti-angiogenic” means that the peptides of the invention block, inhibit or reduce the process of angiogenesis, or the process by which new blood vessels form by developing from pre-existing vessels. Such peptides can block angiogenesis by blocking or reducing any of the steps involved in angiogenesis, including the steps of (1) dissolution of the membrane of the originating vessel, (2) migration and proliferation of the endothelial cells, and (3) formation of the new vascular tube by the migrating cells.

In particular, the peptides of the invention block, inhibit or reduce VEGF-induced stimulation of endothelial cell activation or proliferation, as may be detected or measured using any one or more of the assays described herein or in the available literature. For instance, the ability of the disclosed peptides to inhibit or reduce VEGF-induced stimulation may be measured by incubating the disclosed peptides in the presence of VEGF and monitoring any reduction in the proliferation or survival of bovine retinal endothelial cells (BRE) or human umbilical vein endothelial cells (HUVEC) as described herein. Other measures of endothelial cell stimulation may also be used, including detecting the effect of the peptides on the expression of one or more antiapoptotic proteins such as BcI-2 or A1 (see Gerber et al., 1998, J. Biol. Chem. 273(21): 133313-16), or the effect of the peptides on the phosphorylation or dephosphorylation of VEGF signal transducing proteins such as Akt (see Gerber et al, 1998, 273(46): 30336-43).

The peptides of the invention also block, inhibit or reduce VEGF binding to the KDR, as may be detected or measured using the disclosed mini peptide display technology, or any known competitive or non-competitive KDR binding assay. In this regard, labeled minicells or any other cell expressing a peptide of the invention may be used to detect or measure binding of the disclosed peptides to the KDR. The present invention also encompasses labeled peptide derivatives of any of the peptides disclosed herein, wherein the peptide is conjugated or complexed to a detectable label such as a radioactive, fluorescent, luminescent, proteogenic, immunogenic or any other suitable molecule.

The term “peptide” as used in the present invention is equivalent with the term “polypeptide” and refers to a molecule comprising a sequence of at least six amino acids, but does not refer to polypeptide sequences of whole, native or naturally occurring proteins. Thus, the peptides of the invention have at least six amino acids and preferably not more than about 100, 75, 50, 40, 30, 25, 20 or 15 amino acids. Most preferred peptides of the invention will have at least about six amino acids but no more than about 12 amino acids.

Based on homology alignment of the peptides identified using mini peptide display technology with KDR blocking peptides of the prior art, the inventors identified a consensus sequence of LPPHSS (SEQ ID No. 1) that provides the core sequence for a novel family of peptides having substantially improved anti-angiogenic properties. This core consensus sequence was further expanded by homology alignment to include at least one or more of the N-terminal amino acids ATS, and/or at least one or more of the C-terminal amino acids QSP, creating expanded consensus sequences of ATSLPPHSS (SEQ ID No. 10), LPPHSSQSP (SEQ ID No. 13) and ATSLPPHSSQSP (SEQ ID No. 16).

Peptides comprising the amino acid sequence of SEQ ID No. 16 in particular have been shown to demonstrate a significantly lower IC₅₀ of about 40 micromolar versus about 200 micromolar when compared to previously known peptides. Accordingly, peptides of the present invention demonstrate the functional attributes of anti-angiogenic activity, and may further block or reduce VEGF binding to KDR at a concentration of less than about 200 micromolar, more preferably at a concentration less than about 175, 150, 125, 100 or 75 micromolar, and most preferably at a concentration less than about 50 micromolar.

Preferred peptides of the present invention include but are not limited to the following peptide sequences:

### LPPHSS (SEQ ID No. 1)

### SLPPHSS (SEQ ID No. 2)

### LPPHSSQ (SEQ ID No. 3)

### SLPPHSSQ (SEQ ID No. 4)

### TSLPPHSS (SEQ ID No. 5)

### LPPHSSQS (SEQ ID No. 6)

### TSLPPHSSQ (SEQ ID No. 7)

### SLPPHSSQS (SEQ ID No. 8)

### TSLPPHSSQS (SEQ ID No. 9)

### ATSLPPHSSS (SEQ ID No. 10)

### ATSLPPHSSQ (SEQ ID No. 11)

### ATSLPPHSSQS (SEQ ID No. 12)

### LPPHSSQSP (SEQ ID No. 13)

### SLPPHSSQSP (SEQ ID No. 14)

### TSLPPHSSQSP (SEQ ID No. 15)

### ATSLPPHSSQSP (SEQ ID No. 16)

### ATSLPPHSSLQT (SEQ ID No. 17)

### ATSLPPHSSQSPL (SEQ ID No. 18)

### ATSLPPHSSQSPRAL (SEQ ID No. 19)

### SLPPRALQ (SEQ ID No. 20)

### TSLPPRAL (SEQ ID No. 21)

### LPPRALQS (SEQ ID No. 22)

### TSLPPRALQ (SEQ ID No. 23)

### SLPPRALQS (SEQ ID No. 24)

### TSLPPRALQS (SEQ ID No. 25)

### ATSLPPRAL (SEQ ID No. 26)

### ATSLPPRALQ (SEQ ID No. 27)

### ATSLPPRALQS (SEQ ID No. 28)

### LPPRALQSP (SEQ ID No. 29)

### SLPPRALQSP (SEQ ID No. 30)

### TSLPPRALQSP (SEQ ID No. 31)

### ATSLPPRALQSP (SEQ ID No. 32)

### WLPPHSS (SEQ ID No. 33)

### ATWLPPHSSQSP (SEQ ID No. 34)

### WLPPRAL (SEQ ID No. 35)

### ATWLPPRALQSP (SEQ ID No. 36)

### PSQSSHPPLSTA (SEQ ID No. 37)

Peptides of the invention may “comprise” the disclosed sequences, i.e., where the disclosed sequence is part of a larger peptide sequence that may or may not provide additional functional attributes to the disclosed peptide, such as enhanced solubility and/or stability, fusion to marker proteins for monitoring or measuring peptide activity or binding, larger peptides comprising immunogenic or antigenic peptides, etc. Preferred peptides of the invention may be described as including sequences “consisting essentially” of the disclosed sequences in addition to extraneous sequences which do not affect the anti-angiogenic activity and functional binding properties of the peptides. Alternatively, the peptides of the invention may consist only of the disclosed peptide sequences.

The sequences of the core peptides can be modified via conservative substitutions and/or by chemical modification or conjugation to other molecules in order to enhance parameters like solubility, serum or plasma stability, etc, while retaining anti-angiogenic activity and binding to KDR. In particular, the peptides of the invention may be acetylated at the N-terminus and/or amidated at the C-terminus, or conjugated, complexed or fused to molecules that enhance serum stability, including but not limited to albumin, immunoglobulins and fragments thereof, transferrin, lipoproteins, liposomes, α-2-macroglobulin and α-1-glycoprotein, polyethylene glycol and dextran. Such molecules are described in detail in U.S. Pat. No. 6,762,169, which is herein incorporated by reference in its entirety. Peptides and functional conservative variants having either L-amino acids or D-amino acids are included, particularly D-amino acid peptides having the reverse core sequences (retro inverso peptides), such as the peptide having amino acid sequence SEQ ID No. 37, shown above. Retro inverso peptides have been shown to be more suitable for pharmaceutical development, while they retain biological in vitro activity, they are also serum protease resistant, resulting in enhanced in vivo biological activity. In addition, the peptide may be modified by reducing one or more of the peptide bands to enhance stability (Pennington “solid-phase synthesis of peptides containing the CH₂NH reduced band surrogate” in Molecular Biology, ed M. W. Pennington and B. M. Dunn 35 (1994) 241-247 Humana Press Inc., Totowa, N.J.).

Conservative amino acid substitutions may be made with either naturally or non-naturally occurring amino acids. Appropriate conservative substitutions may be determined using any known scoring matrix or standard similarity comparison, including but not limited to the substitutions described in the following: Bordo and Argos, Suggestions for ‘Safe’ Residue Substitutions in Site-Directed Mutagensis, J. Mol. Biol. 217 (1991) 721-729; Taylor, The Classification of Amino Acid Conservation, J. Theor. Biol. 119 (1986) 205-218; French and Robson, J. Mol. Evol. 19 (1983) 171; Pearson, Rapid and Sensitive Sequence Comparison with FASTP and FASTA, in Methods in Enzymology, ed. R. Doolittle (ISBN 0-12-182084-X, Academic Press, San Diego) 183 (1990) 63-98; and Johnson and Overington A Structural Basis for Sequence Comparisons: An Evaluation of Scoring Methodologies, J. Mol. Biol. (1993) 233, 716-738; and U.S. Pat. No. 5,994,125, each of which is herein incorporated by reference in its entirety. Some exemplary conservative substitutions based on chemical properties are included in Table 1 below.

TABLE 1
Exemplary Conservative Amino Acid Substitutions

| Interchangeable Amino Acids | Properties |
|---|---|
| Lysine (K), Arginine (R), Histidine (H), | basic, large, polar, |
| Ornithine, Homoarginine | hydrophilic, positively |
| | charged |
| Aspartic Acid (D), Glutamic Acid (E), | small, polar, acidic, |
| Asparagrine (N), Glutamine (Q) | negatively charged |
| Isoleucine (I), Leucine (L), Methionine | hydrophobic, large, |
| (M), Phenylalanine (F), Tryptophan (W), | polar or nonpolar |
| Tyrosine (Y), Valine (V), Cysteine (C), | |
| Noravaline, Homoalanine | |
| Alanine (A), Glycine (G), Serine (S), | small, nonpolar, |
| Threonine (T), Cysteine (C), Asparagrine | uncharged, hydrophilic |
| (N), Glutamine (Q), Homoalanine | |
| Phenylalanine (F), Tryptophan (W), | aromatic |
| Tyrosine (Y), Histidine (H) | |
| Proline (P), Amino isobutyric acid (Aib), | cyclic, bending |
| Cycloleucine | |

The present invention also encompasses antibodies that specifically bind to the peptides disclosed herein. Exemplary antibodies include polyclonal, monoclonal, humanized, fully human, chimeric, bispecific, and heteroconjugate antibodies. Monoclonal antibodies may be prepared using hybridoma methods, such as those described by Kohler and Milstein, Nature 256:495 (1975), which is herein incorporated by reference. Alternatively, lymphocytes may be immunized in vitro. The immunizing agent will typically include the peptide or a fusion protein thereof, further comprising a carrier or adjuvant protein.

Anti-idiotypic antibodies may also be prepared using standard procedures that exhibit properties substantially similar to the peptides as herein described. Such antibodies may therefore be used to inhibit or reduce VEGF-mediated stimulation of endothelial cells in the same manner as the disclosed peptides. Antibodies specific for the disclosed peptides may be labeled and used to detect the peptide, for instance in any of the receptor binding assays described herein. Alternatively, such antibodies may be used to purify recombinantly synthesized peptide.

### Nucleic Acids

The present invention also encompasses isolated nucleic acids encoding the peptides described herein, as well as vectors comprising such nucleic acids for cloning (amplification of the DNA) or for expression. Various vectors are publicly available. The vector may, for example, be in the form of a plasmid, cosmid, viral particle, or phage. Such nucleic acids may be used to produce the peptide substrate, for instance by expressing the nucleic acid in a host cell. It will be understood by those skilled in the art that different nucleic acid sequences may encode the same amino acid sequence due to the degeneracy of the triplet code, and that the invention encompasses all possible nucleic acid sequences coding for the peptides described herein. Such nucleic acids may be synthetically prepared and cloned into any suitable vector using methods that are well known in the art.

Using well known cloning techniques, peptide coding sequences may be fused in frame to a signal sequence to allow secretion by the host cell. Alternatively, such peptides may be produced as a fusion to another protein, and thereafter separated and isolated by the use of a site specific protease. Such systems for producing peptides and proteins are commercially available. It will also be feasible to employ such host cells in methods for detecting expression of KDR by a test cell, or in methods of detecting VEGF activity in a sample, for instance by mixing a test cell or a sample with a host cell expressing a peptide of the invention and detecting binding of said host cell or said peptide or by detecting inhibition of VEGF activity. Suitable host cells include eukaryotic and prokaryotic cells. Vectors containing promoters for protein expression in specific host cells of interest are known and publicly available.

Nucleic acids and expression vectors encoding peptides of the invention may also be used in the therapeutic methods described herein, for instance as gene therapy vehicles to deliver the expressed peptide to the disease site. Suitable vectors are typically viral vectors, including DNA viruses, RNA viruses, and retroviruses (see Scanlon, 2004, Anticancer Res. 24(2A):501-4, for a recent review, which is herein incorporated by reference in its entirety). Controlled release systems, fabricated from natural and synthetic polymers, are also available for local delivery of vectors, which can avoid distribution to distant tissues, decrease toxicity to nontarget cells, and reduce the immune response to the vector (Pannier and Shea, 2004, Mol. Ther. 10(1):19-26).

### Methods of Use

The peptides of the present invention may be used in a variety of methods, including but not limited to methods of detecting KDR expression and methods of detecting and/or inhibiting VEGF/receptor interaction. For instance, the peptides of the invention may be conjugated to radioactive or fluorescent imaging markers for the detection of KDR expressing cells in vivo. Detection of aberrant or increased KDR expression could be an indication of ongoing disease, and could be used to localize malignant tumors or diagnose eye diseases associated with excessive intraocular neovascularization.

The present invention also encompasses methods of using the peptides disclosed herein to screen for compounds that mimic the disclosed peptides (agonists) or prevent the effect of the peptides (antagonists). Screening assays for antagonist drug candidates are designed to identify compounds that bind to KDR, or otherwise interfere with the interaction of the disclosed peptides with KDR. Such screening assays will include assays amenable to high-throughput screening of chemical libraries, making them particularly suitable for identifying small molecule drug candidates. The assays can be performed in a variety of formats, including protein-protein binding assays, biochemical screening assays, immunoassays, and cell-based assays, which are well characterized in the art.

In particular, antagonists may be detected by combining a peptide of the invention and a potential antagonist with membrane-bound or surface-bound KDR or recombinant receptors under appropriate conditions for a competitive inhibition assay. The peptide of the invention can be labeled, such as by radioactivity or fluorescence, such that the number of peptide molecules bound to the receptor can be used to determine the effectiveness of the potential antagonist.

The invention also encompasses methods for reducing VEGF-mediated angiogenesis, and for blocking VEGF binding to KDR or a KDR peptide, comprising contacting a cell expressing kinase domain receptor (KDR) with the peptides described herein such that VEGF-mediated angiogenesis or VEGF binding, respectively, is reduced. In such methods, the KDR or receptor peptide may be contacted with the peptide of the invention in the presence of VEGF or prior to being exposed to VEGF. Either the KDR or the peptide of the invention may be displayed on a synthetic surface, such as in a protein or peptide array. Alternatively, the KDR or KDR peptide may be expressed on the surface of a cell. KDR-expressing cells to be targeted by the methods of the invention can include either or both prokaryotic and eukaryotic cells. Such cells may be maintained in vitro, or they may be present in vivo, for instance in a patient or subject diagnosed with cancer or another angiogenesis-related disease.

The present invention also includes methods of treating a patient diagnosed with an angiogenesis-related disease with a therapeutically effective amount of any of the peptides described herein, comprising administering said peptide to said patient such that said angiogenesis-related disease is reduced or inhibited. Exemplary angiogenesis-related diseases are described throughout this application, and include but are not limited to diseases selected from the group consisting of tumors and neoplasias, hemangiomas, rheumatoid arthritis, atherosclerosis, idiopathic pulmonary fibrosis, vascular restenosis, arteriovenous malformations, meningioma, neovascular glaucoma, psoriasis, angiofibroma, hemophilia joints, hypertrophic scars, Osler-Weber syndrome, pyogenic granuloma, retrolental fibroplasias, scleroderma, trachoma, vascular adhesion pathologies, synovitis, dermatitis, endometriosis, pterygium, diabetic retinopathy, neovascularization associated with corneal injury or grafts, wounds, sores, and ulcers (skin, gastric and duodenal).

In particular, the invention includes methods of treating a patient diagnosed with cancer with a therapeutically effective amount of any of the peptides described herein, comprising administering said peptide to said patient such that spread of said cancer is reduced or inhibited. Cancers treatable by the methods of the present invention include all solid tumor and metastatic cancers, including but not limited to those selected from the group consisting of kidney, colon, ovarian, prostate, pancreatic, lung, brain and skin cancers.

The present invention also includes methods of treating a patient diagnosed with an angiogenesis-associated eye disease with a therapeutically effective amount of any of the peptides described herein, comprising administering said peptide to said patient such that said eye disease is reduced or inhibited. Such eye diseases include any eye disease associated with abnormal intraocular neovascularization, including but not limited to retinopathy of prematurity, diabetic retinopathy, retinal vein occlusion, and macular degeneration.

The present invention also includes methods of treating a patient diagnosed with an angiogenesis-associated inflammatory condition with a therapeutically effective amount of any of the peptides described herein, comprising administering said peptide to said patient such that said inflammatory condition is reduced or inhibited. Such inflammatory conditions or diseases include any inflammatory disorder associated with expression of VEGF and activation of cells by VEGF, including but not limited to all types of arthritis and particularly rheumatoid arthritis and osteoarthritis, asthma, pulmonary fibrosis and dermatitis.

### Pharmaceutical Formulations

For pharmaceutical uses, the compounds of the present invention may be used in combination with a pharmaceutically acceptable carrier, and can optionally include a pharmaceutically acceptable diluent or excipient. The present invention thus also provides pharmaceutical compositions suitable for administration to a subject. The carrier can be a liquid, so that the composition is adapted for parenteral administration, or can be solid, i.e., a tablet or pill formulated for oral administration. Further, the carrier can be in the form of a nebulizable liquid or solid so that the composition is adapted for inhalation. When administered parenterally, the composition should be pyrogen free and in an acceptable parenteral carrier. Active compounds can alternatively be formulated or encapsulated in liposomes, using known methods.

The pharmaceutical compositions of the invention comprise an effective amount of one or more peptides of the present invention in combination with the pharmaceutically acceptable carrier. The compositions may further comprise other known drugs suitable for the treatment of the particular disease being targeted. An effective amount of the compound of the present invention is that amount that blocks, inhibits or reduces VEGF stimulation of endothelial cells compared to that which would occur in the absence of the compound; in other words, an amount that decreases the angiogenic activity of the endothelium, compared to that which would occur in the absence of the compound. The effective amount (and the manner of administration) will be determined on an individual basis and will be based on the specific therapeutic molecule being used and a consideration of the subject (size, age, general health), the condition being treated (cancer, arthritis, eye disease, etc.), the severity of the symptoms to be treated, the result sought, the specific carrier or pharmaceutical formulation being used, the route of administration, and other factors as would be apparent to those skilled in the art. The effective amount can be determined by one of ordinary skill in the art using techniques as are known in the art. Therapeutically effective amounts of the compounds described herein can be determined using in vitro tests, animal models or other dose-response studies, as are known in the art.

The pharmaceutical compositions of the invention may be prepared, packaged, or sold in formulations suitable for oral, rectal, vaginal, parenteral, topical, pulmonary, intranasal, buccal, ophthalmic, intrathecal or another route of administration. Other contemplated formulations include projected nanoparticles, liposomal preparations, and immunologically based formulations.

Liposomes are completely closed lipid bilayer membranes which contain entrapped aqueous volume. Liposomes are vesicles which may be unilamellar (single membrane) or multilamellar (onion-like structures characterized by multiple membrane bilayers, each separated from the next by an aqueous layer). The bilayer is composed of two lipid monolayers having a hydrophobic “tail” region and a hydrophilic “head” region. In the membrane bilayer, the hydrophobic (nonpolar) “tails” of the lipid monolayers orient toward the center of the bilayer, whereas the hydrophilic (polar) “heads” orient toward the aqueous phase.

The liposomes of the present invention may be formed by any of the methods known in the art. Several methods may be used to form the liposomes of the present invention. For example, multilamellar vesicles (MLVs), stable plurilamellar vesicles (SPLVs), small unilamellar vesicles (SUV), or reverse phase evaporation vesicles (REVs) may be used. Preferably, however, MLVs are extruded through filters forming large unilamellar vesicles (LUVs) of sizes dependent upon the filter size utilized. In general, polycarbonate filters of 30, 50, 60, 100, 200 or 800 nm pores may be used. In this method, disclosed in Cullis et al., U.S. Pat. No. 5,008,050, relevant portions of which are incorporated by reference herein, the liposome suspension may be repeatedly passed through the extrusion device resulting in a population of liposomes of homogeneous size distribution.

For example, the filtering may be performed through a straight-through membrane filter (a Nuclepore polycarbonate filter) or a tortuous path filter (e.g. a Nuclepore Membrafil filter (mixed cellulose esters) of 0.1 μm size), or by alternative size reduction techniques such as homogenization. The size of the liposomes may vary from about 0.03 to above about 2 microns in diameter; preferably about 0.05 to 0.3 microns and most preferably about 0.1 to about 0.2 microns. The size range includes liposomes that are MLVs, SPLVs, or LUVs.

Lipids which can be used in the liposome formulations of the present invention include synthetic or natural phospholipids and may include phosphatidylcholine (PC), phosphatidylethanolamine (PE), phosphatidylserine (PS), phosphatidylglycerol (PG), phosphatidic acid (PA), phosphatidylinositol (PI), sphingomyelin (SPM) and cardiolipin, among others, either alone or in combination, and also in combination with cholesterol. The phospholipids useful in the present invention may also include dimyristoylphosphatidylcholine (DMPC) and dimyristoylphosphatidylglycerol (DMPG). In other embodiments, distearoylphosphatidylcholine (DSPC), dipalmitoylphosphatidylcholine (DPPC), or hydrogenated soy phosphatidylcholine (HSPC) may also be used. Dimyristoylphosphatidylcholine (DMPC) and diarachidonoylphosphatidylcholine (DAPC) may similarly be used.

During preparation of the liposomes, organic solvents may also be used to suspend the lipids. Suitable organic solvents for use in the present invention include those with a variety of polarities and dielectric properties, which solubilize the lipids, for example, chloroform, methanol, ethanol, dimethylsulfoxide (DMSO), methylene chloride, and solvent mixtures such as benzene:methanol (70:30), among others. As a result, solutions (mixtures in which the lipids and other components are uniformly distributed throughout) containing the lipids are formed. Solvents are generally chosen on the basis of their biocompatability, low toxicity, and solubilization abilities.

To encapsulate the peptide(s) of the inventions into the liposomes, the methods described in Chakrabarti et al. U.S. Pat. No. 5,380,531, relevant portions of which are incorporated by reference, herein may be modified for use with the peptide(s) of the present invention.

Liposomes containing the amino acid and peptide formulations of the present invention may be used therapeutically in mammals, especially humans, in the treatment of a number of disease states or pharmacological conditions which require sustained release formulations as well as repeated administration. The mode of administration of the liposomes containing the agents of the present invention may determine the sites and cells in the organism to which the peptide may be delivered.

The liposomes of the present invention may be administered alone but will generally be administered in admixture with a pharmaceutical carrier selected with regard to the intended route of administration and standard pharmaceutical practice. The preparations may be injected parenterally, for example, intravenously. For parenteral administration, they can be used, for example, in the form of a sterile aqueous solution which may contain other solutes, for example, enough salts or glucose to make the solution isotonic, should isotonicity be necessary or desired. The liposomes of the present invention may also be employed subcutaneously or intramuscularly. Other uses, depending upon the particular properties of the preparation, may be envisioned by those skilled in the art.

For the oral mode of administration, the liposomal formulations of the present invention can be used in the form of tablets, capsules, lozenges, troches, powders, syrups, elixirs, aqueous solutions and suspensions, and the like. In the case of tablets, carriers which can be used include lactose, sodium citrate and salts of phosphoric acid. Various disintegrants such as starch, lubricating agents, and talc are commonly used in tablets. For oral administration in capsule form, useful diluents are lactose and high molecular weight polyethylene glycols. When aqueous suspensions are required for oral use, the active ingredient is combined with emulsifying and suspending agents. If desired, certain sweetening and/or flavoring agents can be added.

For the topical mode of administration, the liposomal formulations of the present invention may be incorporated into dosage forms such as gels, oils, emulsions, and the like. These formulations may be administered by direct application as a cream, paste, ointment, gel, lotion or the like. For administration to humans in the treatment of disease states or pharmacological conditions, the prescribing physician will ultimately determine the appropriate dosage of the agent for a given human subject, and this can be expected to vary according to the age, weight and response of the individual as well as the pharmacokinetics of the agent used.

Also the nature and severity of the patient's disease state or condition will influence the dosage regimen. While it is expected that, in general, the dosage of the drug in liposomal form will be about that employed for the free drug, in some cases, it may be necessary to administer dosages outside these limits.

The pharmaceutical compositions of the invention further comprise a depot formulation of biopolymers such as biodegradable microspheres. Biodegradable microspheres are used to control drug release rates and to target drugs to specific sites in the body, thereby optimizing their therapeutic response, decreasing toxic side effects, and eliminating the inconvenience of repeated injections. Biodegradable microspheres have the advantage over large polymer implants in that they do not require surgical procedures for implantation and removal.

The biodegradable microspheres used in the context of the invention are formed with a polymer which delays the release of the peptides and maintains, at the site of action, a therapeutically effective concentration for a prolonged period of time.

The polymer can be chosen from ethylcellulose, polystyrene, poly(∈-caprolactone), poly(lactic acid) and poly(lactic acid-co-glycolic acid) (PLGA). PLGA copolymer is one of the synthetic biodegradable and biocompatible polymers that has reproducible and slow-release characteristics. An advantage of PLGA copolymers is that their degradation rate ranges from months to years and is a function of the polymer molecular weight and the ratio of polylactic acid to polyglycolic acid residues. Several products using PLGA for parenteral applications are currently on the market, including Lupron Depot and Zoladex in the United States and Enantone Depot, Decapeptil, and Pariodel LA in Europe (see Yonsei, Med J. 2000 December; 41(6):720-34 for review).

The pharmaceutical compositions of the invention may further be prepared, packaged, or sold in a formulation suitable for nasal administration as increased permeability has been shown through the tight junction of the nasal epithelialium (Pietro and Woolley, The Science behind Nastech's intranasal drug delivery technology. Manufacturing Chemist, August, 2003). Such formulations may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for nasal delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

Another formulation suitable for intranasal administration is a coarse powder comprising the active ingredient and having an average particle from about 0.2 to 500 micrometers. Such a formulation is administered in the manner in which snuff is taken i.e. by rapid inhalation through the nasal passage from a container of the powder held close to the nares.

Formulations suitable for nasal administration may, for example, comprise from about as little as 0.1% (w/w) and as much as 100% (w/w) of the active ingredient, and may further comprise one or more of the additional ingredients described herein.

The compounds of the present invention can be administered acutely (i.e., during the onset or shortly after events leading to inflammation), or can be administered during the course of a degenerative disease to reduce or ameliorate the progression of symptoms that would otherwise occur. The timing and interval of administration is varied according to the subject's symptoms, and can be administered at an interval of several hours to several days, over a time course of hours, days, weeks or longer, as would be determined by one skilled in the art. A typical daily regime can be from about 0.01 μg/kg body weight per day, from about 1 mg/kg body weight per day, from about 10 mg/kg body weight per day, from about 100 mg/kg body weight per day.

The compounds of the invention may be administered intravenously, orally, intranasally, intraocularly, intramuscularly, intrathecally, or by any suitable route in view of the peptide, the peptide formulation and the disease to be treated. Peptides for the treatment of inflammatory arthritis can be injected directly into the synovial fluid. Peptides for the treatment of solid tumors may be injected directly into the tumor. Peptides for the treatment of skin diseases may be applied topically, for instance in the form of a lotion or spray. Intrathecal administration, i.e. for the treatment of brain tumors, can comprise injection directly into the brain. Alternatively, peptides may be coupled or conjugated to a second molecule (a “carrier”), which is a peptide or non-proteinaceous moiety selected for its ability to penetrate the blood-brain barrier and transport the active agent across the blood-brain barrier. Examples of suitable carriers are disclosed in U.S. Pat. Nos. 4,902,505; 5,604,198; and 5,017,566, which are herein incorporated by reference in their entirety.

An alternative method of administering peptides of the present invention is carried out by administering to the subject a vector carrying a nucleic acid sequence encoding the peptide, where the vector is capable of directing expression and secretion of the peptide. Suitable vectors are typically viral vectors, including DNA viruses, RNA viruses, and retroviruses. Techniques for utilizing vector delivery systems and carrying out gene therapy are known in the art (see Lundstrom, 2003, Trends Biotechnol. 21(3):117-22, for a recent review).

The following examples are provided to describe and illustrate the present invention. As such, they should not be construed to limit the scope of the invention. Those in the art will well appreciate that many other embodiments also fall within the scope of the invention, as it is described herein above and in the claims.

### EXAMPLES

### Example 1

### Identification of Novel Human VEGF Receptor KDR Binding Peptides by Minicell Panning

Methods

A minicell display library comprising random 30-mer oligonucleotides genetically fused to the gene encoding the 17K antigen of Rickettsia rickettsii in the vector pBS (Bluescript) was constructed essentially as described in U.S. patent application 20030105310, which is herein incorporated by reference in its entirety. The library was transformed into E. coli DS410, and transformed cells were grown in a 250 mL culture overnight in rich medium (Terrific Broth). Minicells were purified by differential centrifugation at 9.3 K rpm.

An ELISA-based binding assay for minicell screening was performed as follows:
1. Costar high binding plate 3361 was coated with 5 μg/ml KDR (R&D systems, 357-KD) diluted with 100 mM sodium bicarbonate 30 mM sodium carbonate pH 9.5 coating buffer-50 μl/well. Coating buffer was added alone to two wells as negative control wells.
2. Plate was incubated at 4° C. over-weekend with slight rotation.
3. Next morning: Minicell random library aliquot (10% of pellet) was resuspended in 1 ml PBS. 1 μl Bodipy was added and minicells were stained 10 min while rotating at room temperature. The sample was spun 1 min at 13000 rpm and the pellet was washed 3 times for 5 min with 900 μl PBS with rotation at room temperature. The sample was spun 1 min at 13000 rpm and the pellet resuspended in 560 μl PBS for assay.
4. Unbound KDR was removed from high binding plate to new plate to conserve material.
5. The plate washed once briefly with 200 μl PBS.
6. Labeled minicells added: the minicells were diluted 1:1 with appropriate PBS buffer prepared at 2 fold the concentration of the eventual wash condition (i.e., PBS, PBS with 500 mM NaCl, PBS with 1M NaCl, PBS+0.2% NP−40, PBS+0.02% SDS) and loaded 50 μl/well with 0.1% BSA and 25 μg/ml kanamycin. Minicells were added to control wells as well.
7. The plate was sealed and incubated 4° C. overnight as above (total incubation=18 hrs).
8. Unbound minicells were removed to a new plate to save.
9. The plate was washed 3 times for 1 min with 200 μl of appropriate buffer-PBS, PBS with 250 mM NaCl, PBS with 500 mM NaCl, PBS+0.1% NP−40, PBS+0.01% SDS. 50 μl PBS/well was added and plate was incubated three hours at 4° C.
10. Plate was viewed under microscope at 20× and 40× magnification for labeled minicells.
11. Minicell DNA was extracted from positive wells via phenol-chloroform and transformed into competent DH5alpha cells.
12. Colonies were isolated and cultured in 5 mL LB+100 μg/ml Amp overnight at 37° C.
13. DNA was miniprepped from 1.5 mL of culture via the Qiagen method and processed for sequencing.
14. Sequences were compared to literature for sequences having significant homology.

Homology Analysis

Six clones were obtained and their sequences were compared to sequences disclosed in the following two papers:
Binetruy-Toumaire R, Demangel C, Malavaud B, Vassy R, Rouyre S, Kraemer M, Plouet J, Derbin C, Perret G, Mazie J C., 2000, Identification of a peptide blocking vascular endothelial growth factor (VEGF)-mediated angiogenesis, EMBO J. 19(7): 1525-33. Lu D, Shen J, Vil M D, Zhang H, Jimenez X, Bohlen P, Witte L, Zhu Z., 2003, Tailoring in vitro selection for a picomolar affinity human antibody directed against vascular endothelial growth factor receptor 2 for enhanced neutralizing activity, J. Biol. Chem. 278(44):43496-507.

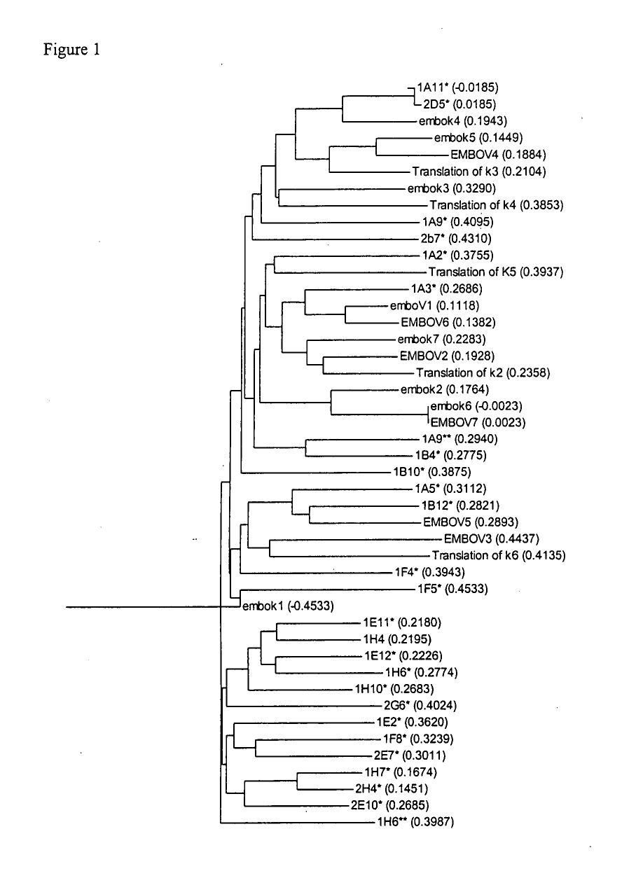

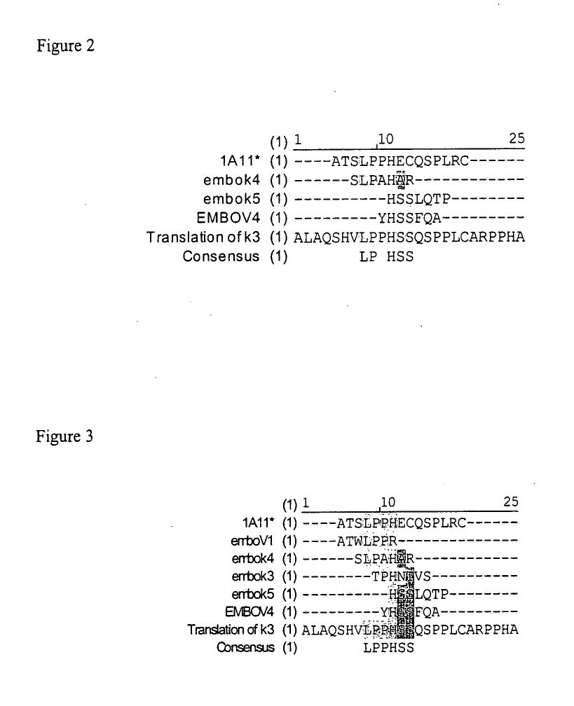

Binetruy-Toumaire et al. used immobilized KDR to screen a phage display library. Lu et al. used a phage display library to further define the fine binding specificities of two fully human neutralizing KDR-specific antibodies. As shown in FIG. 1, By comparing the clones identified by minicell display screening with the peptides disclosed in the two papers referenced above, a series of subgroups was identified (see FIG. 1, a phylogenetic tree generated by custalW using Vector NTI). Of particular interest is the subgroup at the top of the alignment tree, comprising the peptides: EmboK4 (SEQ ID No. 38), EmboK5 (SEQ ID No. 39) and EmboV4 (SEQ ID No. 40) from the paper by Binetruy-Toumaire et al., the two peptides 1A11 and 2D5 (which have the same sequence (SEQ ID No. 41) and therefore will be considered as one) from the paper by Lu et al., and the clone K3 (SEQ ID No. 42) obtained by minicell display technology. The alignment of these peptides is shown in FIG. 2.

The high level of sequence homology between the peptide sequences in FIG. 2 suggested that the K3 peptide or partial fragments of this peptide would have anti-angiogenic properties. Further homology searching with the sequence of this peptide revealed another pocket of homology between K3 and two of the peptides disclosed by Binetruy-Toumaire et al., EmboV1 (SEQ ID No. 43) and EmboK3 (SEQ ID No. 44). The final alignment of all of these peptides is shown in FIG. 3. This alignment revealed the existence of a consensus sequence that is highly conserved among all of the peptides, LPPHSS. While Binetruy-Tournaire et al. discussed the relevance of the LPP sequence for biological activity and mentioned the presence of the HSS sequence in two of the isolated peptides, the combination of both these subsequences together in a single peptide was not disclosed. Nevertheless, in view of the alignment of the sequences and the comparison to the K3 peptide identified using minicell display technology, the present inventors predicted that a peptide with the sequence LPPHSS (SEQ ID No. 1) would have anti-angiogenic properties substantially different and more useful than either of the two isolated sequences by themselves.

In addition, the homology alignment revealed two further regions of consensus. The region ATS that is present in the amino terminal portion of the peptide 1A11 is partially conserved in the EmboV1 (see FIG. 2). Further, the serine residue is present in alignment in EmboK4. Accordingly, the present inventors also predicted that this region would contribute anti-angiogenic properties, and that a peptide with the sequence ATSLPPHSS (SEQ ID No. 10) would have anti-angiogenic properties substantially different and more useful than either of the three isolated sequences alone. The other region of homology covers the subsequence QSP, present in the C-terminal region of peptide 1A11 and in the peptide K3. In addition, the serine is conserved in the peptide EmboK3. Accordingly, the present inventors also predicted that this region would contribute anti-angiogenic properties, and that a peptide with the sequence ATSLPPHSSQSP (SEQ ID No. 16) would have anti-angiogenic properties substantially different and more useful than any of the four isolated sequences alone.

### Example 2

### Characterization of Anti-Angiogenic Activity of KDR Binding Peptides In Vitro

Methods

The following peptides were synthesized to test for anti-angiogenic activities in vitro and in vivo:

| | | |
|---|---|---|
| ST100,037 | LPPHSS | (SEQ ID No. 1) |
| ST100,038 | ATSLPPHSSQSP | (SEQ ID No. 16) |
| ST100,039 | LPPHSSQSP | (SEQ ID No. 13) |
| ST100,040 | Biot-LPPHSSQSP | (SEQ ID No. 13) |

In addition, the following variants of ST100,038 were synthesized using D-amino acids as opposed to L-amino acids to test the effect of the modification on activity and serum stability:

| | | |
|---|---|---|
| ST100,045 | ATSLPPHSSQSP | (SEQ ID No. 16) |
| ST100,059 | PSQSSHPPLSTA | (SEQ ID No. 37) |
| | (retro inverso | |
| | peptide) | |

The anti-angiogenic activities of the peptides were tested by measuring the level of inhibition of VEGF mediated survival/proliferation of bovine retinal endothelial cells (BRE), a standard cell line used to test anti-angiogenic compounds. Cells were maintained in Cambrex EGM-2MV medium. On day one cells were starved for either 6 hours or overnight, and thereafter trypsinized and plated in a 96 well plate in 100 μl of Invitrogen OptiMem+1% fetal bovine serum. A 100 μl aliquot of Invitrogen OptiMem+1% fetal bovine serum was then added to the wells in addition to, where appropriate, VEGF to a final concentration of 25 ng/ml and the various peptides to final concentration of 12.5, 25, 50 and 100 μg/ml. After 72 hours incubation, the number of live cells in each well was determined using the WST-1 assay (Roche).

Table 2 reports the amount of WST-1-induced calorimetric change measured at 440 nm. The data points for each treatment are averaged and presented underneath the peptide name. The VEGF+/− wells are averaged and presented next to the correspondent definition. The Student's t-test values between the peptide treated wells and the VEGF only wells are calculated in the column next to the average.

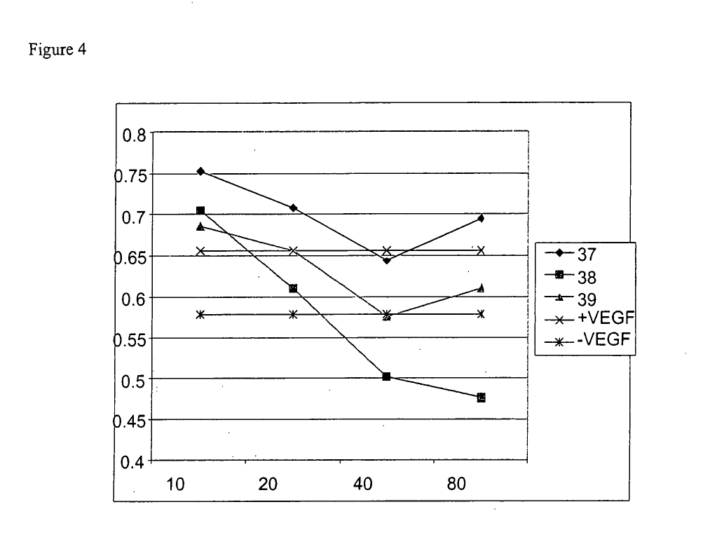

The average of the 3 wells for each data point is graphed in FIG. 4, and indicates how increasing concentrations of peptide decrease the amount of WST-1 and therefore the number of live cells. Student's t-test analysis of the data reveals that these decreases are statistically significant for the two highest concentrations of ST100,038, which appears to be the most active peptide as postulated. Concentrations above 25 μg/ml completely abolished the statistically significant VEGF-induced increase in WST-1 value and actually resulted in even lower values than observed in cells without VEGF stimulation. The most likely explanation is that the peptide inhibits the stimulation of the cells by the growth factors (VEGF) present in the medium.

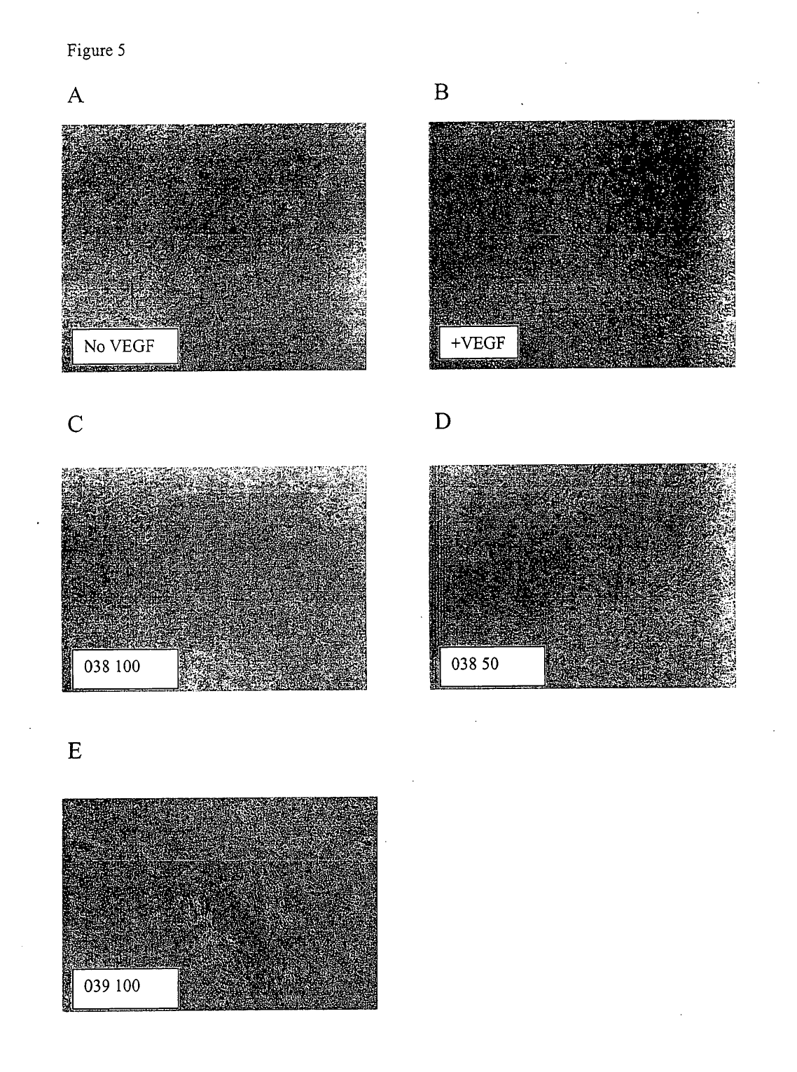

FIG. 5 depicts photomicrographs showing examples of the number and morphology of cells exposed to various treatments. Of particular note is the well treated with 100 μg/ml of ST100,038, which contains very few cells. The few cells that are present show signs of apoptosis (cell death). This is in contrast to the positive control (cells treated with VEGF) and is similar to the cells that received no VEGF.

TABLE 2

| | | ST100,037 | ST100,037 | ST100,037 | ST100,038 | ST100,038 | ST100,038 |
|---|---|---|---|---|---|---|---|
| | | 0.052 | 0.053 | 0.056 | 0.053 | 0.048 | 0.051 |
| Peptide | 100.00 | 0.692 | 0.697 | 0.694 | 0.466 | 0.447 | 0.511 |
| concentration | | | | | | | |
| Peptide | 50.000 | 0.636 | 0.659 | 0.633 | 0.479 | 0.496 | 0.489 |
| concentration | | | | | | | |
| Peptide | 25.000 | 0.716 | 0.713 | 0.695 | 0.598 | 0.625 | 0.590 |
| concentration | | | | | | | |
| Peptide | 12.500 | 0.780 | 0.730 | 0.748 | 0.669 | 0.701 | 0.719 |
| concentration | | | | | | | |
| | +VEGF | 0.717 | 0.679 | 0.672 | 0.623 | 0.626 | 0.704 |
| | −VEGF | 0.606 | 0.608 | 0.608 | 0.594 | 0.565 | 0.569 |
| | | 0.051 | 0.052 | 0.052 | 0.052 | 0.052 | 0.054 |

| | | ST100,038 | ST100,039 | ST100,039 | ST100,039 |
|---|---|---|---|---|---|
| | | 0.051 | 0.052 | 0.049 | 0.051 |
| Peptide | 100.00 | 0.481 | 0.659 | 0.588 | 0.581 |
| concentration | | | | | |
| Peptide | 50.000 | 0.546 | 0.605 | 0.593 | 0.530 |
| concentration | | | | | |
| Peptide | 25.000 | 0.624 | 0.717 | 0.687 | 0.564 |
| concentration | | | | | |
| Peptide | 12.500 | 0.732 | 0.701 | 0.735 | 0.621 |
| concentration | | | | | |
| | +VEGF | 0.657 | 0.488 | 0.676 | 0.715 |
| | −VEGF | 0.556 | 0.550 | 0.567 | 0.563 |
| | | 0.052 | 0.051 | 0.052 | 0.052 |

| | | |
|---|---|---|
| ST100,037 | ST100,038 | ST100,039 |

| | average | Ttest | average | Ttest | average | Ttest |
|---|---|---|---|---|---|---|
| 100.00 | 0.694 | 0.707 | 0.476 | 0.000 | 0.609 | 0.292 |
| 50.000 | 0.643 | 0.929 | 0.503 | 0.001 | 0.576 | 0.082 |
| 25.000 | 0.708 | 0.454 | 0.609 | 0.208 | 0.656 | 0.995 |
| 12.500 | 0.753 | 0.246 | 0.705 | 0.188 | 0.686 | 0.504 |
| +VEGF | 0.656 | | | | | |
| −VEGF | 0.5786 | 0.00302 | | | | |

The experiment was repeated using higher concentrations of peptide, to a maximum concentration of 200 μg/ml. This required the addition of a substantial amount of water (the peptides are soluble in water and are maintained as stock solution of 2 mg/ml). Therefore, we tested whether the addition of water itself would have any inhibitory effects. As before, BRE were maintained in Cambrex EGM-2MV medium. On day one, cells were starved for either 6 hours or overnight, and thereafter trypsinized and plated in a 96 well plate in 100 μl of OptiMem+1% fetal bovine serum. A 100 μl aliquot of OptiMem+1% fetal bovine serum was then added to the wells, further containing, where appropriate, VEGF to a final concentration of 25 ng/ml and the various peptides to final concentration of 25, 50, 100 and 200 μg/ml. After 72 hours incubation, the amount of live cells in each well was measured using the WST-1 assay (Roche).

Table 3 reports the amount of WST-1-induced calorimetric change measured at 440 nm. The data points for each treatment are averaged and presented underneath the peptide name. The VEGF+/− wells are averaged and presented next to the correspondent definition. The Student's t-test values between the peptide treated wells and the VEGF only wells are calculated in the column next to the average.

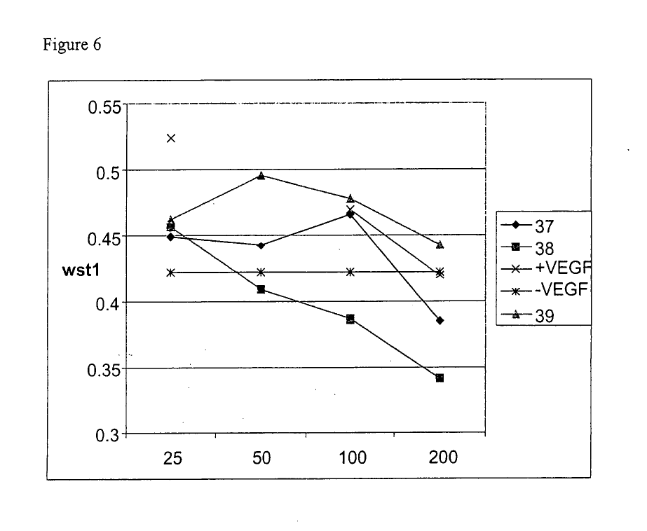

The average of the 3 wells for each data point is graphed in FIG. 6. Student's t-test analysis of the data revealed that the WST-1 decreases are statistically significant for all concentrations of ST100,038 which as previously shown is the most active peptide. Concentrations above 25 μg/ml resulted again in values lower than seen with no VEGF stimulation.

TABLE 3

| | ST100,037 | ST100,037 | ST100,037 | ST100,038 | ST100,038 |
|---|---|---|---|---|---|
| 200 | 0.384 | 0.401 | 0.372 | 0.341 | 0.336 |
| 100 | 0.449 | 0.486 | 0.462 | 0.406 | 0.399 |
| 50 | 0.427 | 0.438 | 0.462 | 0.403 | 0.413 |
| 25 | 0.439 | 0.432 | 0.476 | 0.450 | 0.434 |
| +VEGF | 0.410 | 0.430 | 0.419 | 0.423 | 0.481 |
| −VEGF | 0.334 | 0.338 | 0.393 | 0.393 | 0.448 |
| | 10% water | 10% water | 10% water | 10% water | 5% water |
| | ST100,038 | ST100,038 | ST100,039 | ST100,039 | ST100,039 |
| 200 | 0.348 | 0.343 | 0.416 | 0.453 | 0.459 |
| 100 | 0.382 | 0.361 | 0.451 | 0.517 | 0.465 |
| 50 | 0.365 | 0.458 | 0.489 | 0.507 | 0.491 |
| 25 | 0.461 | 0.482 | 0.411 | 0.495 | 0.480 |
| +VEGF | 0.504 | 0.528 | 0.521 | 0.530 | 0.519 |
| −VEGF | 0.487 | 0.454 | 0.457 | 0.463 | 0.461 |
| | 5% water | 5% water | No water | No water | No water |

| [conc] | | ST100,037 | TTEST | | ST100,038 | TTEST | | ST100,039 | TTEST |
|---|---|---|---|---|---|---|---|---|---|
| 200 | | 0.386 | 0.035 | | 0.342 | 0.000 | | 0.443 | 0.191 |
| 100 | | 0.466 | 0.896 | | 0.387 | 0.017 | | 0.478 | 0.804 |
| 50 | | 0.442 | 0.002 | | 0.410 | 0.001 | | 0.496 | 0.004 |
| 25 | | 0.449 | 0.006 | | 0.457 | 0.001 | | 0.462 | 0.035 |
| +VEGF | 10% water | 0.421 | | 5% water | 0.504 | 0.034584669 | No water | 0.525 | |
| −VEGF | | 0.365 | | | 0.463 | | | 0.462 | |

### Example 3

### Stability Studies of Peptides in 1% or 10% Serum

Sample Preparation

A stock solution of 1 mM peptide dissolved in water was made. The stock was then diluted to 100 μM in either OptiMem medium+100 units/ml of penicillin and 100 μg/ml streptomycin sulfate+1% fetal bovine serum or in OptiMem+Pen/Strep+10% fetal bovine serum. The diluted samples were placed in a 24-well tissue culture plate in an incubator at 37 degrees. Aliquots of 50-100 μl were removed at 4, 6, 18, 24, 48 and 72 hrs and frozen at −70° C. until analysis.

Analysis by LC/MS

Samples of 20 μl were separated on a C18 reverse phase column (4.8×250 mm) with a gradient of acetonitrile/water 0.1% TFA and analyzed using a single quad mass spectrometer. Singly or multiply charged peaks were detected depending on the mass of the peptide. Peptide degradation was determined in two ways: loss of peak area in the chromatogram produced using the mass spectrometer as the detector and loss of the main peak in the mass spectrum with simultaneous appearance of a peak(s) from a breakdown product.

Results

In 1% serum, 25% of ST100,038 was lost at 18 hours, 33% at 24 hours, 60% at 48 hours and 85% at 72 hrs. In 10% serum, 50% of the peptide was degraded in 4 hours, 65% by 6 hours and none remained at 18 hours. All cleavages appeared to be N-terminal to serine. In 1% serum, the peptide degraded to smaller peptides which continued to persist through the 72 hour time point. In 10% serum, even these smaller peptides were barely detectable by 48 hours.

In consideration of the relatively limited stability in serum of ST100,03, ST100,045 and ST100,059 were synthesized. ST100,045 has the same sequence of ST10,038 but it is made with D-amino acids. ST100,059 is the D-amino acid peptide with an inverted sequence (retro-inverso peptide). They were tested for serum stability using the protocol described above and did not degrade under any of the tested conditions.

While it is generally understood in the art that D-amino acid peptides are more stable in serum, replacing L- with D-amino acid peptides does not automatically generate an active and stable peptide. Our own data described below with the ST100,038, ST100,045 and ST100,059 series revealed that only the retro inverso ST100,059 is still biologically active and serum stable, while ST100,045, which contains D-amino acids and the same sequence as ST100,038, is somewhat less biologically active than its L-amino acid counterpart.

### Example 4

### Characterization of Anti-Angiogenic Activity of D-Amino Acid Peptide Derivatives

The activity of ST100,045 was then compared to that of ST100,038. As before, BRE were maintained in Cambrex EGM-2MV medium. On day one, cells were starved for either 6 hours or overnight, trypsinized and then plated in a 96 well plate in 100 μl of OptiMem+1% fetal bovine serum. Afterwards, 100 μl of OptiMem+1% fetal bovine serum were added to the wells in addition to, where appropriate, VEGF to a final concentration of 25 ng/ml and the various peptides to final concentration of 5, 12.5, 25 and 50 μg/ml. After 72 hours incubation, the number of live cells in each well was measured with the WST-1 assay (Roche).

Table 4 reports the amount of WST-1 induced colorimetric change measured at 440 nm. The data points for each treatment (50, 25, 12.5 and 5 microgram/ml, respectively) are averaged and presented underneath the peptide name. The VEGF+/− wells are averaged and presented next to the correspondent definition. The Student's t-test values between the peptide treated wells and the VEGF only wells are calculated in the column next to the average.

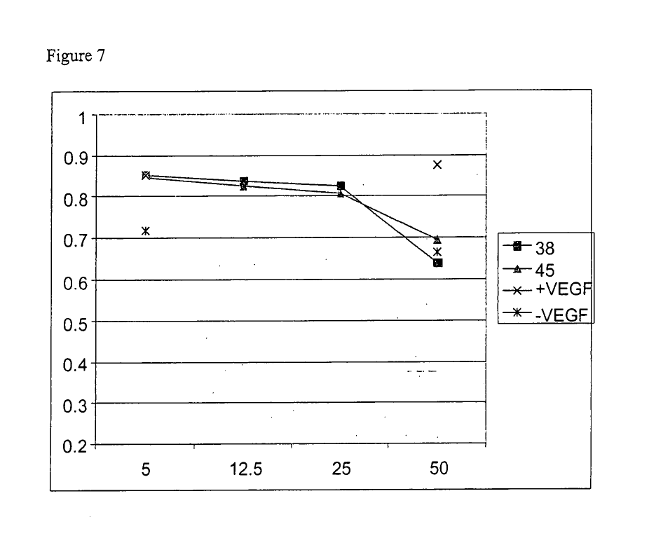

The average of the 3 wells for each data point is graphed in FIG. 7. Student's t-test analysis of the data reveals that WST-1 decreases are statistically significant for the highest concentrations of ST100,038 and ST100,045. Concentrations above 25 μg/ml again resulted in values lower than no VEGF stimulation.

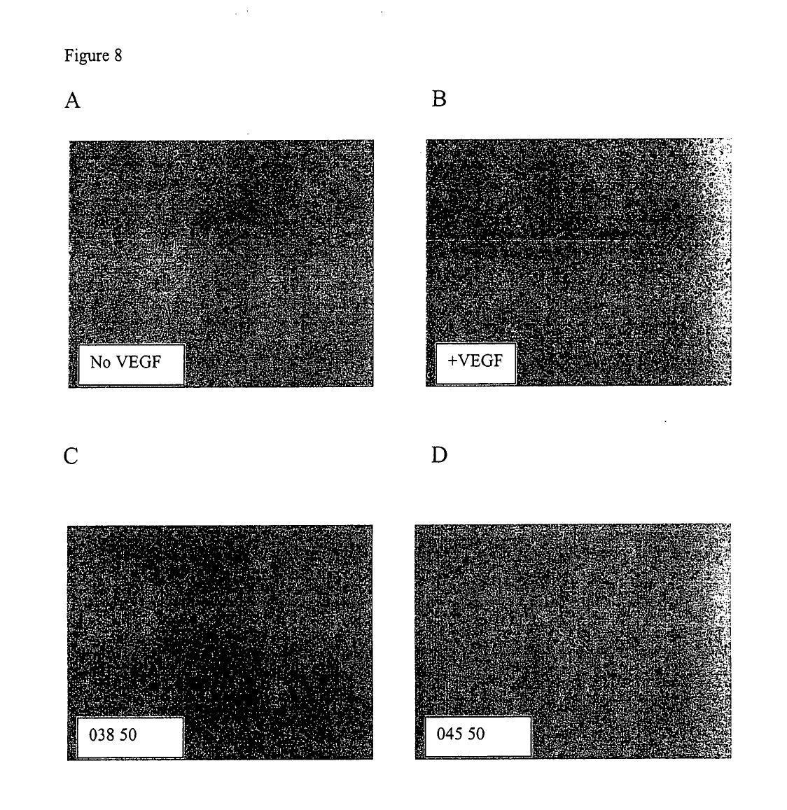

The photomicrographs in FIG. 8 show examples of the number and morphology of cells exposed to various treatments. Particularly noteworthy are the wells treated with either 50 μg/ml of ST100,038 or ST100,045, where there are very few cells. Further, the few cells that are present show sign of apoptosis (cell death). This is in contrast to the positive control (cells treated with VEGF) and is similar to cells that received no VEGF.

| | | ST100,038 | ST100,038 | ST100,038 | ST100,038 | ST100,045 | ST100,045 | ST100,045 | ST100,045 |
|---|---|---|---|---|---|---|---|---|---|
| peptide | 50 | 0.644 | 0.654 | 0.627 | 0.629 | 0.704 | 0.715 | 0.696 | 0.661 |
| concentration | | | | | | | | | |
| peptide | 25 | 0.783 | 0.856 | 0.83 | 0.835 | 0.838 | 0.827 | 0.847 | 0.717 |
| concentration | | | | | | | | | |
| peptide | 12.5 | 0.8 | 0.837 | 0.86 | 0.85 | 0.873 | 0.886 | 0.827 | 0.715 |
| concentration | | | | | | | | | |
| peptide | 5 | 0.858 | 0.855 | 0.839 | 0.856 | 0.835 | 0.901 | 0.907 | 0.744 |
| concentration | | | | | | | | | |
| | +VEGF | 0.816 | 0.881 | 0.861 | 0.856 | 0.882 | 0.855 | 0.882 | 0.887 |
| | −VEGF | 0.703 | 0.734 | 0.697 | 0.739 | 0.632 | 0.674 | 0.645 | 0.711 |

| | ST100,038 | TTEST | ST100,045 | TTEST |
|---|---|---|---|---|
| 50 | 0.6385 | 7.36E−06 | 0.694 | 0.000113 |
| 25 | 0.826 | 0.025091 | 0.80725 | 0.068351 |
| 12.5 | 0.83675 | 0.811575 | 0.82525 | 0.242552 |
| 5 | 0.852 | 0.919896 | 0.84675 | 0.470384 |
| +VEGF | 0.865 | 0.00023 | | |
| −VEGF | 0.692 | | | |

### Example 5

### Characterization of Anti-Angiogenic Activity of Peptides on Adherent Cells

In this experiment, the peptides were added to the wells after the cells had adhered overnight. On day one cells were starved for 6 hours, they were then trypsinized and plated in 96 well plate in 100 μl of OptiMem+1% fetal bovine serum. The morning after, when cells had already adhered, 100 μl of OptiMem+1% fetal bovine serum were added to the wells in addition, where appropriate, VEGF to a final concentration of 25 ng/ml and the various peptides to final concentration of 10, 30, 50 and 75 μg/ml. After 72 hours incubation, the amount of live cells in each well was measured with the WST-1 assay (Roche).

Table 5 reports the amount of WST-1 induced colorimetric change measured at 440 nm. The data points for each treatment (75, 50, 30 or 10 μg/ml, respectively) are averaged and presented underneath the peptide name. The VEGF+/− wells are averaged and presented next to the correspondent definition. The Student's t-test values between the peptide treated wells and the VEGF only wells are calculated in the column next to the average.

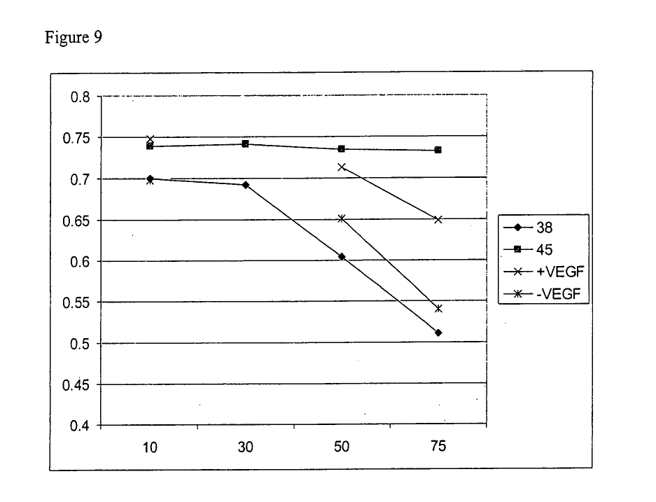

The average of the 3 wells for each data point is graphed in FIG. 9. Student's t-test analysis of the data reveals that WST-1 decreases are statistically significant for the 2 highest concentrations of ST100,038. Concentrations above 25 μg/ml again resulted in values lower than those seen with no VEGF stimulation. However, no effect was observed with ST100,045. This represents an interesting difference in the activity of ST100,038 and ST100,045, with only ST100,038 being able to inhibit the growth of BRE after they have adhered to the plate. The following experiments confirmed this difference.

Table 6 reports the repeat of adding ST100,038 and ST100,045 to BRE before they adhered. The data points for each treatment are averaged and presented underneath the peptide name. The VEGF+/− wells are averaged and presented next to the correspondent definition. The Student's t-test values between the peptide treated wells and the VEGF only wells are calculated in the column next to the average.

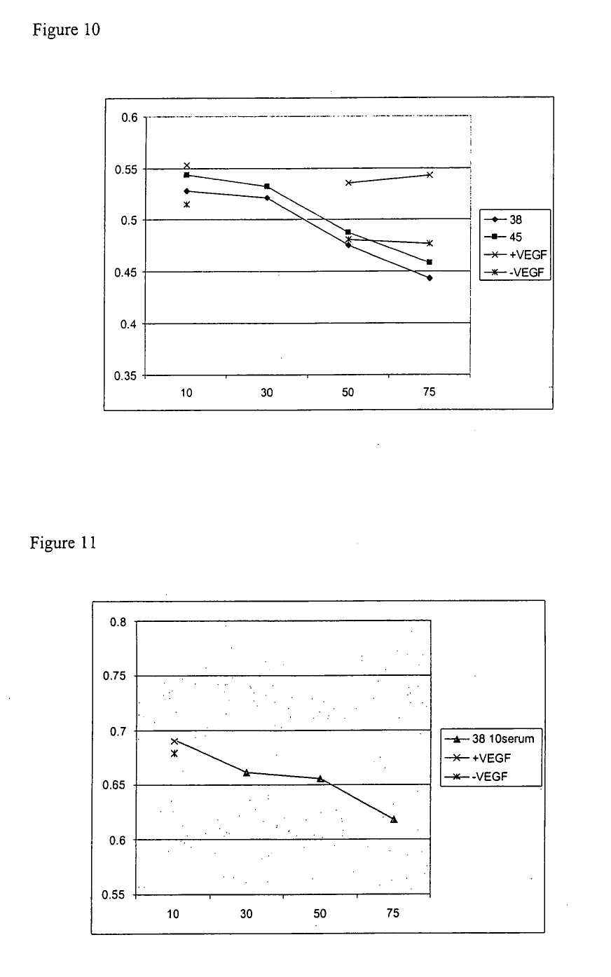

As previously seen, both peptides inhibited BRE growth and survival (see FIG. 10). In addition, ST100,038 was tested in the treatment of cells grown in OptiMem+10% fetal bovine serum, where it inhibited BRE growth and survival under these conditions as well (Table 6, columns 8-11, rows B-C-D-E) (FIG. 11).

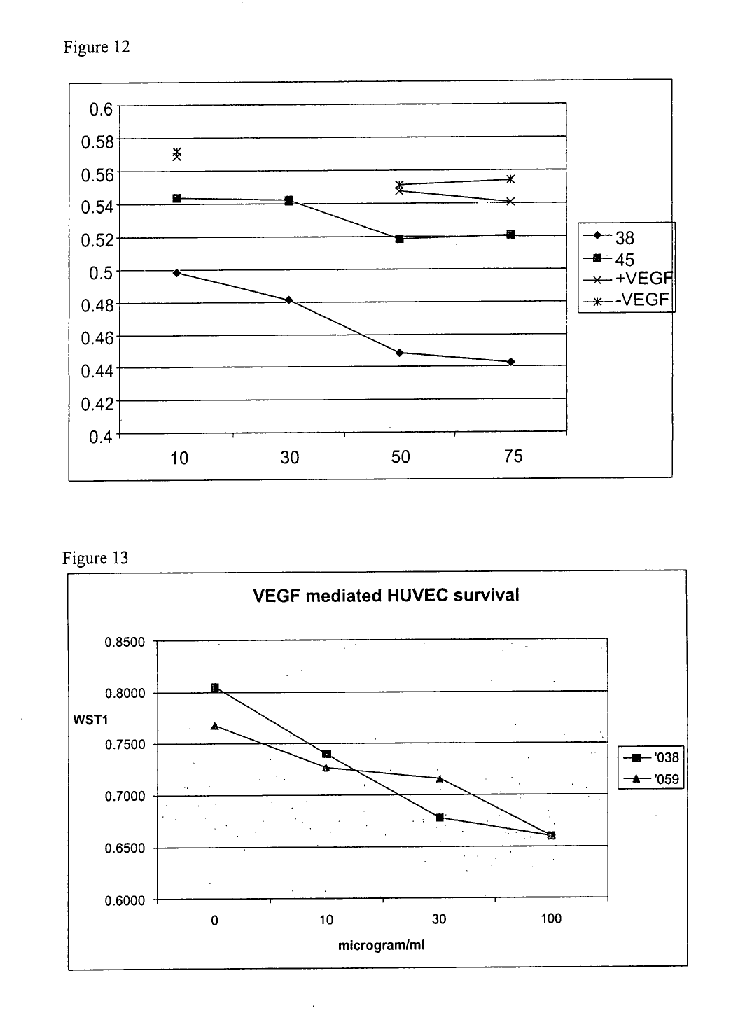

Table 7 reports the repeat experiment of adding ST100,038 and ST100,045 to BRE after they adhered. To adjust for diluent, to the indicated control wells, 10% water or 5% water or no water was added. The VEGF+/− wells are averaged and presented next to the correspondent definition. The Student's t-test values between the peptide treated wells and the VEGF only wells are calculated in the column next to the averaged. As previously seen, only ST100,038 substantially inhibited BRE growth and survival when added after the cells have adhered (see FIG. 12).

TABLE 5

| | | ST100,038 | ST100,038 | ST100,038 | ST100,045 | ST100,045 | ST100,045 | | | |
|---|---|---|---|---|---|---|---|---|---|---|
| peptide | 75 | 0.5 | 0.52 | 0.511 | 0.735 | 0.745 | 0.717 | | | |
| concentration | | | | | | | | | | |
| peptide | 50 | 0.579 | 0.626 | 0.609 | 0.723 | 0.734 | 0.746 | | | |
| concentration | | | | | | | | | | |
| peptide | 30 | 0.671 | 0.702 | 0.705 | 0.736 | 0.744 | 0.744 | | | |
| concentration | | | | | | | | | | |
| peptide | 10 | 0.693 | 0.691 | 0.717 | 0.729 | 0.739 | 0.75 | | | |
| concentration | | | | | | | | | | |
| | +VEGF | 0.597 | 0.684 | 0.667 | 0.707 | 0.696 | 0.737 | 0.749 | 0.748 | 0.747 |
| | −VEGF | 0.54 | 0.561 | 0.521 | 0.642 | 0.643 | 0.667 | 0.68 | 0.704 | 0.708 |
| | | +10% water | +10% water | +10% water | +5% water | +5% water | +5% water | no water | no water | no water |

| | | ST100,038 | TTEST | | ST100,045 | TTEST | | |
|---|---|---|---|---|---|---|---|---|
| 75 | | 0.510333 | 0.00697246 | | 0.732333 | 0.04074937 | | |
| 50 | | 0.604667 | 0.00412138 | | 0.734333 | 0.2063236 | | |
| 30 | | 0.692667 | 0.00705959 | | 0.741333 | 0.07094875 | | |
| 10 | | 0.700333 | 0.00470346 | | 0.739333 | 0.22791311 | | |
| +VEGF | +10% water | 0.64933333 | | +5% water | 0.713333 | | no water | 0.748 |
| −VEGF | | 0.54066667 | | | 0.650667 | | | 0.69733333 |

TABLE 6

| | ST100,038 | ST100,038 | ST100,038 | ST100,045 | ST100,045 |
|---|---|---|---|---|---|
| 75 | 0.448 | 0.444 | 0.438 | 0.455 | 0.458 |
| 50 | 0.476 | 0.469 | 0.479 | 0.492 | 0.488 |
| 30 | 0.519 | 0.517 | 0.528 | 0.527 | 0.531 |
| 10 | 0.517 | 0.546 | 0.521 | 0.545 | 0.534 |
| +VEGF | 0.543 | 0.546 | 0.539 | 0.539 | 0.523 |
| −VEGF | 0.486 | 0.472 | 0.472 | 0.474 | 0.47 |
| | | ST100,038 | ST100,038 | ST100,038 | ST100,038 |
| | ST100,045 | 10% serum | 10% serum | 10% serum | 10% serum |
| 75 | 0.462 | 0.615 | 0.602 | 0.637 | 0.734 |
| 50 | 0.481 | 0.663 | 0.646 | 0.658 | 0.669 |
| 30 | 0.538 | 0.657 | 0.642 | 0.684 | 0.667 |
| 10 | 0.551 | 0.688 | 0.702 | 0.685 | 0.696 |
| +VEGF | 0.545 | 0.563 | 0.557 | 0.54 | 0.673 |
| −VEGF | 0.497 | 0.525 | 0.503 | 0.517 | 0.669 |

| | | | | | | ST100,038 | | |
|---|---|---|---|---|---|---|---|---|
| | ST100,038 | TTEST | | ST100,045 | TTEST | 10% serum | TTEST | |
| | 0.443333 | 9.63E−06 | 0.013335 | 0.458333 | 7.96E−06 | 0.618 | 0.041244 | 0.69 |
| | 0.474667 | 0.001065731 | 0.047769 | 0.487 | 0.002644638 | 0.655667 | 0.202991 | |
| | 0.521333 | 0.124296956 | 0.084254 | 0.532 | 0.048485157 | 0.661 | 0.314028 | |
| | 0.528 | 0.090232102 | 0.212582 | 0.543333 | 0.304558785 | 0.691667 | 0.944808 | 0.679333 |
| +VEGF | 0.542667 | 0.000203792 | | 0.535667 | 0.006583776 | 0.553333 | 0.710056 | |
| −VEGF | 0.476667 | | | 0.480333 | | 0.515 | | |

TABLE 7

| | | ST100,038 | ST100,038 | ST100,038 | ST100,045 | ST100,045 | ST100,045 | | | |
|---|---|---|---|---|---|---|---|---|---|---|
| peptide | 75 | 0.445 | 0.442 | 0.44 | 0.52 | 0.522 | 0.52 | | | |
| concentration | | | | | | | | | | |
| peptide | 50 | 0.447 | 0.445 | 0.453 | 0.518 | 0.525 | 0.512 | | | |
| concentration | | | | | | | | | | |
| peptide | 30 | 0.479 | 0.481 | 0.483 | 0.532 | 0.538 | 0.555 | | | |
| concentration | | | | | | | | | | |
| peptide | 10 | 0.484 | 0.505 | 0.506 | 0.535 | 0.548 | 0.547 | | | |
| concentration | | | | | | | | | | |
| | +VEGF | 0.531 | 0.544 | 0.547 | 0.545 | 0.545 | 0.552 | 0.564 | 0.549 | 0.594 |
| | −VEGF | 0.545 | 0.558 | 0.56 | 0.572 | 0.544 | 0.537 | 0.563 | 0.561 | 0.591 |
| | | +10% water | +10% water | +10% water | +5% water | +5% water | +5% water | no water | no water | no water |

| | ST100,038 | TTEST | ST100,045 | TTEST | |
|---|---|---|---|---|---|
| 75 | 0.442333 | 4.33E−05 | 0.520667 | 0.0156552 | |
| 50 | 0.448333 | 7.81E−06 | 0.518333 | 0.0027964 | |
| 30 | 0.481 | 0.002689547 | 0.541667 | 0.1407923 | |
| 10 | 0.498333 | 0.00933564 | 0.543333 | 0.1379588 | |
| +VEGF | 0.540667 | | 0.547333 | | 0.569 |
| −VEGF | 0.554333 | | 0.551 | | 0.571667 |

### Example 6

### Characterization of Anti-Angiogenic Activity of Retro-Inverso Peptide Derivative

The above results showed that both ST100,038 and ST100,045 can inhibit VEGF mediated BRE growth and survival, with ST100,038 being efficacious in a wider set of conditions. We decided therefore to generate ST100,059, an D-amino acid peptide having the inverted sequence of ST100,038. This peptide represents the “retro-inverso” version of 038. There is evidence in the literature that such peptides fit exactly the same binding site in the receptor while being much more stable.

In this experiment we tested the activity of ST100,059 in comparison to that of ST100,038. Human umbilical vein endothelial cells (HUVEC) were maintained in Cambrex EGM-2MV medium. On day one, cells were trypsinized and plated in a 96 well plate in 100 μl of OptiMem+2% fetal bovine serum. Alternately, 100 μl of OptiMem was then added to the wells in addition to, where appropriate, VEGF to a final concentration of 10 ng/ml and the various peptides to final concentrations of 10, 30 or 100 μg/ml. After 72 hours incubation, the number of live cells in each well was measured with the WST-1 assay (Roche).

Table 8 reports the amount of WST-1 induced colorimetric change measured at 440 nm. The VEGF+/− wells are averaged and presented next to the correspondent definition. The Student's t-test values between the peptide treated wells and the VEGF only wells are calculated in the column next to the average. The standard deviations (STD) are calculated in the column next to the Student's t-test.

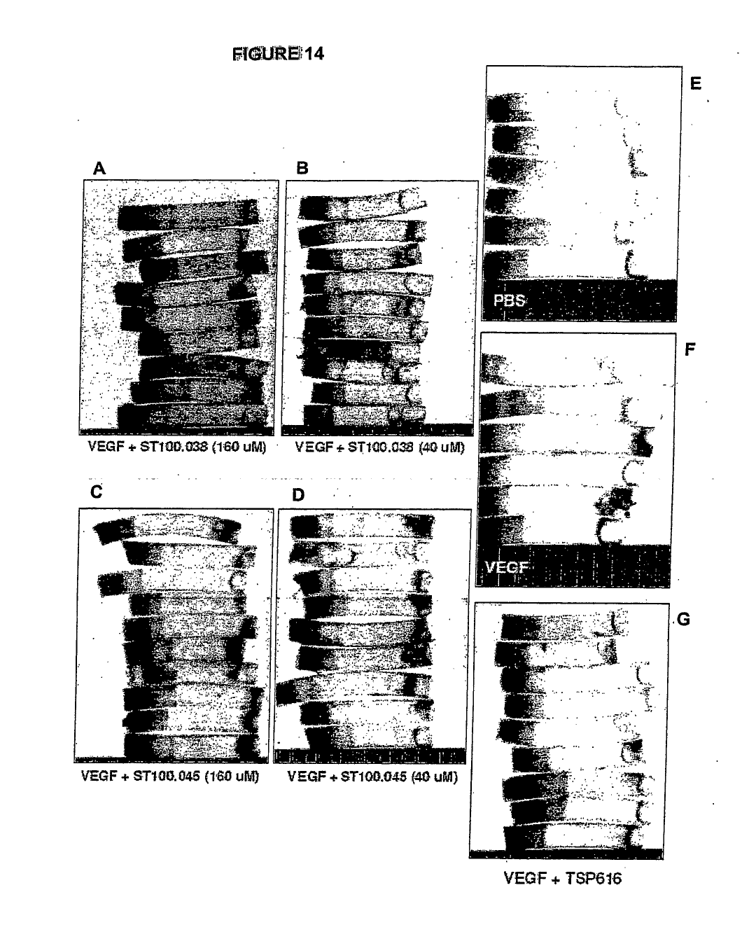

The average of the 4 wells for each data point is graphed (FIG. 13). Student's t-test analysis of the data reveals that the WST-1 decreases are statistically significant for the 2 highest concentrations of ST100,038 and for the highest concentration of ST100,059. Accordingly, ST100,059 has an inhibitory activity similar to ST100,038.

TABLE 8

| | ST100,038 | ST100,038 | ST100,038 | ST100,038 | ST100,059 | ST100,059 | ST100,059 | ST100,059 |
|---|---|---|---|---|---|---|---|---|
| −VEGF | 0.517 | 0.528 | 0.552 | 0.544 | 0.499 | 0.511 | 0.505 | 0.582 |
| +VEGF | 0.85 | 0.785 | 0.805 | 0.779 | 0.823 | 0.601 | 0.704 | 0.798 |
| 10 | 0.764 | 0.765 | 0.785 | 0.646 | 0.73 | 0.733 | 0.721 | 0.721 |
| 30 | 0.693 | 0.568 | 0.716 | 0.733 | 0.741 | 0.694 | 0.681 | 0.746 |
| 100 | 0.633 | 0.676 | 0.686 | 0.646 | 0.681 | 0.666 | 0.639 | 0.656 |

| | ST100,038 | TTEST | STD | ST100,059 | TTEST | STD |
|---|---|---|---|---|---|---|
| −VEGF | 0.5353 | | 0.0157 | 0.5243 | | 0.0388 |
| +VEGF | 0.8048 | | 0.0321 | 0.7315 | | 0.1010 |
| 10 | 0.7400 | 0.1184 | 0.0634 | 0.7263 | 0.2387 | 0.0062 |
| 30 | 0.6775 | 0.0204 | 0.0748 | 0.7155 | 0.1551 | 0.0328 |
| 100 | 0.6603 | 0.0004 | 0.0249 | 0.6605 | 0.0068 | 0.0176 |

### Example 7

### Characterization of Anti-Angiogenic Activity of KDR Binding Peptides In Vivo

The peptides of the invention were also tested in an in vivo model of anti-angiogenic activity. This model analyzes VEGF-induced angiogenesis as it occurs in angio-reactors filled with Matrigel in wild-type FVB/N mice (Guedez et al., 2003, Am. J. Pathol. 162:1431-1439).

Methods

### 1. Preparation of the Angio-Reactors

Sterile, polyethene tubing (0.14 cm internal diameter) is cut to standard 1 cm lengths using a plexiglass template and single edge razor blade. These tubes are sealed at one end with nail-polish. Into the tubes, 20 μl of Matrigel (growth factor free obtained from BD Biosciences) containing 500 ng/ml VEGF with or without the indicated peptides is injected. After one hour of polymerization of the Matrigel at room temperature the angio-reactors are subcutaneously implanted into both flanks of wild-type FVB/N female mice (8-10 weeks old).

### 2. Determination of Angiogenesis

After 10 days, angiogenesis in the angio-reactors is determined. Mice receive a 100 μl injection of 25 mg/ml of FITC-dextran in phosphate-buffered saline (PBS) via tail vein 20 minutes before collection of the angio-reactors. Quantification of vessel functionality is performed by removal of the Matrigel from the angio-reactors and the fluorescence is measured using a FLUOstar Galaxy microplate reader (excitation 485 nm, emission 520 nm, BMG Labtechnologies GmbH, Germany). The mean relative fluorescence±SD for 10 angio-reactors is determined and statistical analysis performed.

### 3. Schematic Representation of Experiment 1

To be able to perform a statistical analysis of the results of the study, the different arms of the experiments must include 10 angio-reactors in 5 mice. The various groups of animals are listed below in Table 9.

TABLE 9

| | growth | | | |
|---|---|---|---|---|
| Group | factor | inhibitor | concentration | # mice |
| 1 | none | | | 3 |
| 2 | VEGF | none | | 5 |
| 3 | VEGF | TSP 616 | 40 μM | 5 |
| 4 | VEGF | ST100,038 | 40 μM | 5 |
| 5 | VEGF | ST100,038 | 160 μM | 5 |
| 6 | VEGF | ST100,045 | 40 μM | 5 |
| 7 | VEGF | ST100,045 | 160 μM | 5 |

VEGF concentration: 500 ng/ml matrigel (30 μl/angio-reactor)
Thrombospondin peptide 616: SPWSSCSVTCGDGVITRIR (SEQ ID No. 45) (Iruela-Arispe et al., 1999. Circ. 100: 1423-1431).

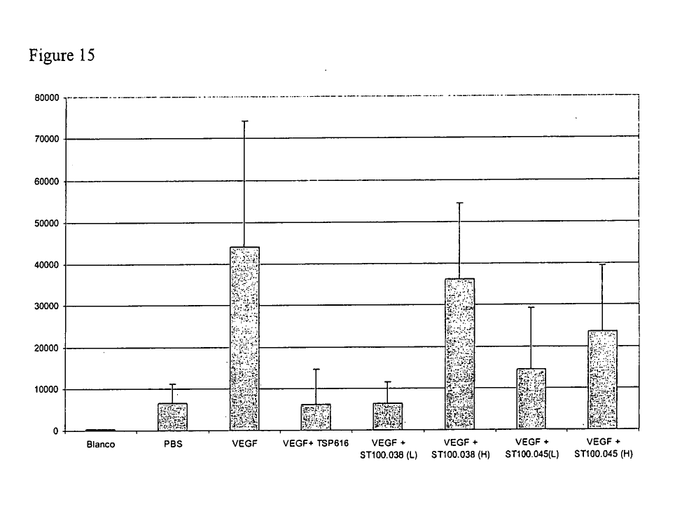

The quantitative results of the experiment are presented in Table 10. The photographs of FIG. 14 show the angioreactors after removal from the mice and represent a qualitative appreciation of the level of angiogenesis. Of the 10 angioreactor in the positive control group (those treated with VEGF alone) only six could be analyzed. Of these six, only three were found to be responding to VEGF (values comparable with previous experiments and above the unstimulated PBS controls). If only those three are used for analysis, it is clear that the lower doses of ST100,038 and ST100,045, i.e., 40 micromolar corresponding to 50 microgram/ml, clearly inhibit VEGF-mediated angiogenesis, with ST100,038 being more active than ST100,045 and bringing the level down to the unstimulated PBS controls and similar to the level obtained with the TSP616 peptide (FIG. 15). It is noteworthy that these results reflect what was seen in vitro using the BRE cells. Thus, 50 μg/ml of ST100,038 was able to completely inhibit VEGF stimulated survival/proliferation.

TABLE 10

| | | | mean | std | sem | TTEST |
|---|---|---|---|---|---|---|
| Blank | blank | 405 | 357 | 62 | 27 | |
| | blank | 306 | | | | |
| | blank | 427 | | | | |
| | blank | 283 | | | | |
| | blank | 363 | | | | |
| PBS | 1 | 3951 | 6583 | 4506 | 1840 | |
| | 2 | 9271 | | | | |
| | 3 | 2199 | | | | |
| | 4 | 13829 | | | | |
| | 5 | 2738 | | | | |
| | 6 | 7507 | | | | |
| VEGF | 7 | 1272 | 22763 | 30152 | 12310 | |
| | | | | | | Using |
| | | | | | | only the |
| | | | | | | values in |
| | | | | | | bold |
| | 8 | 57054 | 44180 | 29945 | | |
| | 9 | 1001 | | | | |
| | 10 | 9951 | | | | |
| | 11 | 65535 | | | | |
| | 12 | 1765 | | | | |
| VEGF + | 13 | 3063 | 6111 | 8412 | 2804 | 0.135115 |
| TSP616 | 14 | 28196 | | | | 0.003992 |
| | 15 | 4316 | | | | |
| | 16 | 4851 | | | | |
| | 17 | 5219 | | | | |
| | 18 | 888 | | | | |
| | 19 | 4395 | | | | |
| | 20 | 2098 | | | | |
| | 21 | 1977 | | | | |
| VEGF + | 22 | 3394 | 6274 | 5173 | 1636 | 0.106173 |
| ST100.038 | 23 | 2133 | | | | 0.001402 |
| 40 uM | 24 | 16940 | | | | |
| | 25 | 2018 | | | | |
| | 26 | 10224 | | | | |
| | 27 | 11716 | | | | |
| | 28 | 5798 | | | | |
| | 29 | 2045 | | | | |
| | 30 | 1795 | | | | |
| | 31 | 6673 | | | | |
| VEGF + | 32 | 17666 | 36271 | 18122 | 6041 | 0.448911 |
| ST100.038 | 33 | 51300 | | | | 0.612271 |
| 160 uM | 34 | 52293 | | | | |
| | 35 | 11917 | | | | |
| | 36 | 25644 | | | | |
| | 37 | 9000 | | | | |
| | 38 | 45234 | | | | |
| | 39 | 65535 | | | | |
| | 40 | 47850 | | | | |
| VEGF + | 41 | 2685 | 14466 | 14636 | 4879 | |
| ST100.045 | 42 | 4491 | | | | 0.038607 |
| 40 uM | 43 | 26585 | | | | |
| | 44 | 14346 | | | | |
| | 45 | 41844 | | | | |
| | 46 | 2738 | | | | |
| | 47 | 4019 | | | | |
| | 48 | 29426 | | | | |
| | 49 | 4056 | | | | |
| VEGF + | 50 | 33376 | 23492 | 15995 | 5058 | |
| ST100.045 | 51 | 8304 | | | | |
| 160 uM | 52 | 11902 | | | | |
| | 53 | 13246 | | | | |
| | 54 | 8764 | | | | |
| | 55 | 32267 | | | | |
| | 56 | 15360 | | | | |
| | 57 | 58010 | | | | |
| | 58 | 35815 | | | | |
| | 59 | 17875 | | | | |

### 4. Schematic Representation of Experiment 2

The various groups of animals tested in experiment 2 are listed below in Table 11.

TABLE 11

| | Growth | | |
|---|---|---|---|
| Group | factor | Inhibitor | # mice |
| 1 | None | | 3 |
| 2 | VEGF | None | 5 |
| 3 | VEGF | TSP 616 | 5 |
| 4 | VEGF | ST100,038 | 5 |
| 5 | VEGF | ST100,059 | 5 |

VEGF concentration: 500 ng/ml matrigel (30 μl/angio-reactor)
Thrombospondin peptide 616: SPWSSCSVTCGDGVITRIR (SEQ ID No. 45) (Iruela-Arispe et al., 1999. Circ. 100: 1423-1431).

The quantitative results of the experiment are presented in Table 12.

TABLE 12

| | | | VEGF + | VEGF + | VEGF + |
|---|---|---|---|---|---|
| | PBS | VEGF | TSP616 | ST 038 | ST 059 |
| | 370 | 3844 | 521 | 789 | 426 |
| | 1325 | 3141 | 769 | 624 | 125 |
| | 429 | 3683 | 484 | 438 | 1596 |
| | 414 | 1008 | 862 | 2060 | 180 |
| | | 1265 | 2335 | 1161 | 3081 |
| | | 3317 | 197 | 981 | 842 |
| | | 5471 | 102 | 288 | 1660 |
| | | 4508 | 232 | 1154 | 174 |
| | | | 213 | 2107 | 179 |
| average | 634.5 | 3279.625 | 635 | 1066.889 | 918.1111 |
| median | 421.5 | 3500 | 484 | 981 | 426 |

Table 13 below contains a Bonferroni's Multiple Comparison Statistical Test of the various group.

Bonferroni's Multiple Comparison Test

| | Mean | | | 95% CI of |
|---|---|---|---|---|
| | Diff. | t | P value | diff |
| pbs vs vegf | −2645 | 4.448 | P < 0.001 | −4430 to −860.1 |
| pbs vs tsp | −0.5 | 0.000857 | P > 0.05 | −1752 to 1751 |
| pbs vs ST 038 | −432.4 | 0.741 | P > 0.05 | −2184 to 1319 |
| pbs vs ST 059 | −283.6 | 0.486 | P > 0.05 | −2035 to 1468 |
| vegf vs tsp | 2645 | 5.605 | P < 0.001 | 1228 to 4061 |
| vegf vs ST 038 | 2213 | 4.69 | P < 0.001 | 796.3 to 3629 |
| vegf vs ST 059 | 2362 | 5.005 | P < 0.001 | 945.1 to 3778 |
| tsp vs ST 038 | −431.9 | 0.9435 | P > 0.05 | −1806 to 942.3 |
| tsp vs ST 059 | −283.1 | 0.6185 | P > 0.05 | −1657 to 1091 |
| ST 038 vs ST 059 | 148.8 | 0.325 | P > 0.05 | −1225 to 1523 |

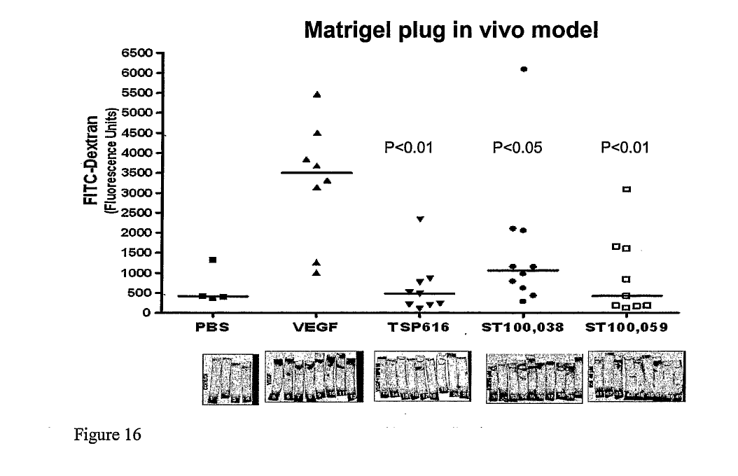

The graph in FIG. 16, summarizing the results by reporting the median of the values, shows that ST100,038 and ST100,059 peptides clearly inhibit VEGF-mediated angiogenesis. ST100,059 is more active than ST100,038 and brings the level of angiogenesis down to that of the unstimulated PBS controls and similar to the level obtained with the TSP616 peptide. It is noteworthy that these results reflect what was seen in vitro using the BRE cells. In addition, comparison of experiments 1 and 2 reveals that the retro-inverso peptide ST100,059 is more active than ST100,045, the peptide generated by simply replacing L-amino acids with D-amino acids.

### Example 8

### Characterization of Anti-Tumor Activity of KDR Binding Peptides In Vivo

The peptides of the invention were tested in an in vivo model of anti-tumor activity. This model compares the growth of subcutaneous B16 melanoma tumor either untreated or treated with either 20 mg/kg, 40 mg/kg, 100 mg/kg daily ip doses of ST100,059.

Methods

Male C57BL/6 mice were obtained with a mean body weight of 20±2 g. Mouse B16-F1 melanoma cells were implanted subcutaneously (5×10⁵ cell per animal). Peptides (formulated in water) were administered ip daily starting the day after injection of cells. Tumors became palpable around 9 days after injection of the cells. Tumors were then measured every 2 days.

The quantitative results of the experiment are presented in Table 14.

TABLE 14

| Group | Mouse # | Day 11 | Day 13 | Day 15 | |
|---|---|---|---|---|---|
| | | | | | Day 17 |
| Control | 1 | 253.12 | 600.00 | 1210.94 | 2025.00 |
| | 2 | 445.50 | 859.63 | 1912.50 | 3159.00 |
| | 3 | 208.25 | 469.63 | 1028.50 | 1913.63 |
| | 4 | 208.25 | 465.75 | 1008.00 | 1787.12 |
| | 5 | 171.50 | 384.75 | 816.75 | 1666.00 |
| | 6 | 330.00 | 620.00 | 1170.00 | 1958.00 |
| | 7 | 173.93 | 541.88 | 1223.31 | 2816.00 |
| | 8 | 253.44 | 520.00 | 1240.40 | 2169.00 |
| | 9 | 196.00 | 518.94 | 1116.00 | 2009.00 |
| | 10 | 250.00 | 510.50 | 1140.50 | 2274.00 |
| | Average | 249.00 | 549.11 | 1186.69 | 2177.68 |
| | STDEV | 83.65 | 128.07 | 285.35 | 467.13 |
| | SEM | 26.45 | 40.50 | 90.24 | 147.72 |
| | | | | | Day 16 |
| Doxorubicin | 1 | 144.00 | 433.50 | 675.00 | 1296.00 |
| | 2 | small | 211.25 | 469.63 | 767.13 |
| | 3 | 169.00 | 325.13 | 633.94 | 1267.50 |
| | 4 | 240.40 | 525.00 | 945.50 | 1759.00 |
| | 5 | 158.44 | 451.25 | 847.00 | 1549.13 |
| | 6 | 196.00 | 405.00 | 756.25 | 1267.50 |
| | 7 | 405.00 | 870.50 | 1350.00 | 1826.00 |
| | 8 | NT | 135.00 | 288.00 | 550.00 |
| | 9 | 189.40 | 380.50 | 730.00 | 1125.00 |
| | 10 | 205.00 | 460.00 | 750.75 | 1206.00 |
| | Average | 213.41 | 419.71 | 744.61 | 1261.33 |
| | STDEV | 82.98 | 197.86 | 282.63 | 397.56 |
| | SEM | 26.24 | 62.57 | 89.38 | 125.72 |
| Group | Mouse # | Day 11 | Day 13 | Day 15 | Day 17 |
| ST 59 (20 mg/kg) | 1 | 393.19 | 700.00 | 1224.00 | 2049.94 |
| | 2 | 320.00 | 526.50 | 1080.00 | 1690.00 |
| | 3 | 210.40 | 610.50 | 940.00 | 1744.00 |
| | 4 | 180.50 | 370.00 | 688.00 | 1236.00 |
| | 5 | 198.00 | 440.00 | 890.50 | 1531.00 |
| | 6 | 204.55 | 580.00 | 910.50 | 1789.00 |
| | 7 | 190.00 | 445.00 | 1050.00 | 1956.00 |
| | 8 | 98.31 | 328.00 | 725.00 | 1362.50 |
| | 9 | 345.00 | 625.00 | 1144.00 | 1739.00 |
| | 10 | 206.06 | 496.25 | 1003.75 | 1452.00 |
| | Average | 234.60 | 512.13 | 965.58 | 1654.94 |
| | STDEV | 89.30 | 118.60 | 171.45 | 257.86 |
| | SEM | 28.24 | 37.50 | 54.22 | 81.54 |
| ST 59 (40 mg/kg) | 1 | 243.19 | 575.00 | 995.00 | 1698.00 |
| | 2 | 272.00 | 508.00 | 936.00 | 1576.88 |
| | 3 | 340.60 | 650.30 | 1190.00 | 1805.00 |
| | 4 | 175.60 | 315.00 | 726.00 | 1320.00 |
| | 5 | 281.00 | 510.30 | 906.00 | 1603.00 |
| | 6 | 128.50 | 324.00 | 682.00 | 1127.00 |
| | 7 | 145.31 | 361.25 | 650.00 | 1028.50 |
| | 8 | 230.50 | 490.30 | 775.00 | 1350.00 |
| | 9 | 310.30 | 618.00 | 1125.00 | 1742.00 |
| | 10 | 150.00 | 325.00 | 705.00 | 1159.00 |
| | Average | 227.70 | 467.72 | 869.00 | 1440.94 |
| | STDEV | 74.54 | 127.83 | 191.32 | 279.89 |
| | SEM | 23.57 | 40.42 | 60.50 | 88.51 |
| ST 59 (100 mg/kg) | 1 | 260.00 | 450.20 | 810.50 | 1549.00 |
| | 2 | NT | NT | small | 239.06 |
| | 3 | 225.50 | 510.00 | 1025.00 | 2029.00 |
| | 4 | 162.00 | 433.50 | 700.00 | 1563.25 |
| | 5 | NT | NT | small | 198.00 |
| | 6 | 270.00 | 450.25 | 786.00 | 1398.00 |
| | 7 | 136.13 | 352.00 | 586.63 | 1164.69 |
| | 8 | 196.00 | 405.00 | 816.75 | 1449.13 |
| | 9 | 167.06 | 442.12 | 752.38 | 1253.56 |
| | 10 | NT | 180.00 | 467.00 | 971.75 |
| | Average | 202.38 | 402.88 | 743.03 | 1181.54 |
| | STDEV | 51.21 | 100.50 | 166.45 | 579.51 |
| | SEM | 16.19 | 31.78 | 52.64 | 183.26 |

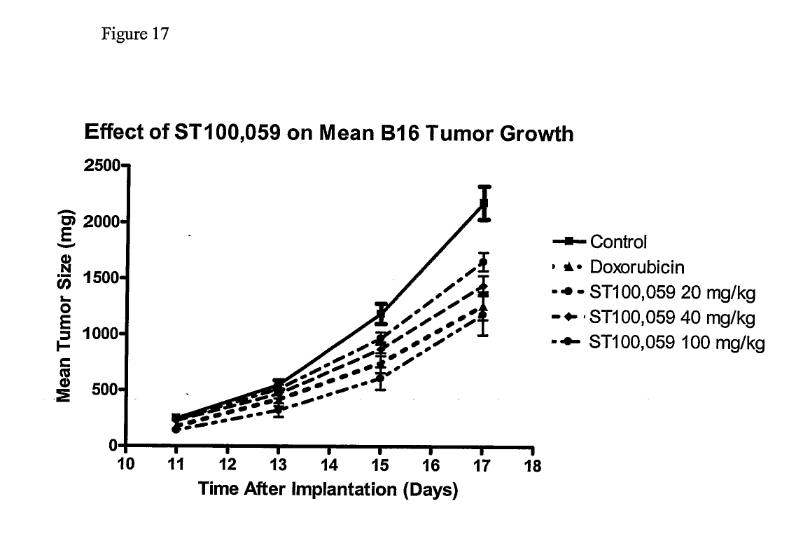

FIG. 17 is a graph comparing inhibition of growth of subcutaneous B16 melanoma tumor in vivo treated with 20 mg/kg, 40 mg/kg or 100 mg/kg daily ip of ST100,059 as compared to untreated controls. ST100,059 is able to inhibit the growth of subcutaneous B16 melanoma tumors in a statistically significant and dose responsive fashion. The testing of ST100,059 in this model was repeated 2 times with similar results.

### Example 9

### Characterization of Anti-Metastatic Activity of KDR Binding Peptides In Vivo

The peptides of the invention were also tested in an in vivo melanoma lung metastasis model. This model compares the number and sizes of mouse B16 melanoma tumor lung metastases in mice either untreated or treated with 100 mg/kg daily ip of ST100,059.

Methods

Male C57BL/6 mice were obtained with a mean body weight of 20±2 g. Mouse B16-F1 melanoma cells were grown in culture, harvested at 85% confluence and inoculated (5×10⁵ cells/mouse) in 100 μl saline via the lateral tail vein.

The mice were sacrificed under anesthesia, on day 14 and the lungs were fixed overnight in Bouin's fluid. Lung metastases were identified and counted in all lobes of lungs.

Schematic Representation of the Experiment

The various groups of animals are listed below in Table 15.

TABLE 15

| Group | | Inhibitor | # mice |
|---|---|---|---|
| 1 | control | | 8 |
| 2 | Doxo | Doxorubicin 5 mg/kg/mouse iv only day 3 | 4 |
| 3 | 059 QD | 100 mg/kg/mouse ip daily | 9 |
| 4 | 059 QOD | 100 mg/kg/mouse ip every 2 days | 9 |

The quantitative results of the experiment are presented in Table 16.

TABLE 16

| control | | 059 QD | | 059 QOD | |
|---|---|---|---|---|---|
| 1 | 20 | 1 | 5 | 1 | 10 |
| 2 | 25 | 2 | 0 | 2 | 5 |
| 3 | 20 | 3 | 30 | 3 | 15 |
| 4 | 15 | 4 | 5 | 4 | 15 |
| 5 | 10 | 5 | 5 | 5 | 0 |
| 6 | 15 | 6 | 0 | 6 | 45 |
| 7 | 25 | 7 | 0 | 7 | 5 |
| 8 | 0 | 8 | 5 | 8 | 10 |
| AVE | 16.25 | | 6.25 | | 13.13 |
| STDEV | 8.35 | | 9.91 | | 13.87 |
| SEM | 2.95 | | 3.50 | | 4.9 |

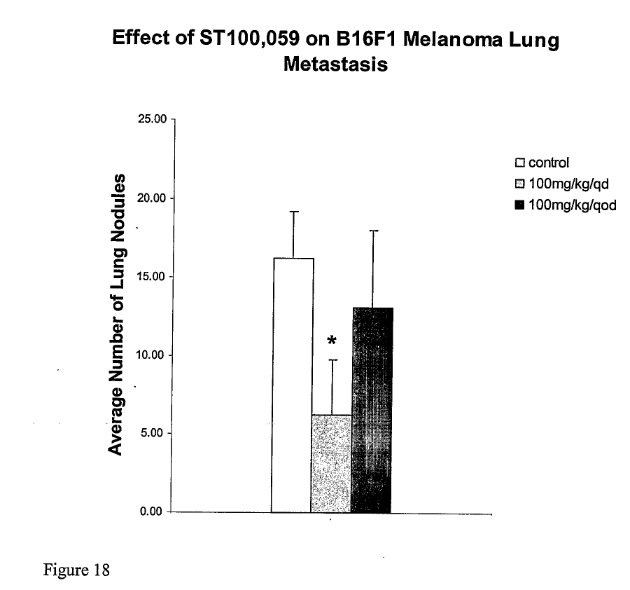

The graph in FIG. 18 summarizing the results shows that ST100,059 clearly reduces the number of lung metastases, with ST100,059 100 mg/kg/mouse ip daily being statistically significant in an ANOVA analysis P<0.05 using Dunne test for multiple comparison if the single outlier with value=30 is not included in the test. The testing of ST100,059 in this model was repeated with similar results.

### Example 10

### Characterization of Anti-Tumor Activity of KDR Binding Peptides In Vivo

The peptides of the invention were tested in an in vivo model of anti-tumor activity. This model compares the growth of the human breast cancer MDA-MB231 tumor xenografts in nude mice treated with 10 mg/kg or 20 mg/kg daily ip of ST100,059, docetaxel or saline.

Methods

Female nude mice (nu/nu) between 5 and 6 weeks of age weighing approximately 20 g were obtained from Harlan, Inc. Animals were implanted subcutaneously (s.c.) by trocar with fragments of human tumors harvested from s.c. grown tumors in nude mice hosts. When the tumors were approximately 60-75 mg in size (about 10-15 days following implantation), the animals were pair-matched into treatment and control groups. Each group contained 8-10 mice, each of which is ear-tagged and followed individually throughout the experiment.

The administration of drugs or controls began the day the animals were pair-matched with tumor size of about 70 mg (Day 1). Mice were weighed and tumor measurements were obtained using calipers twice weekly, starting on Day 1. These tumor measurements were converted to mg tumor weight by the standard formula, (W²×L)/2. Upon termination of the experiment, the mice were weighed, sacrificed and their tumors were excised. The tumors were weighed, and the mean and medium tumor weight per group was calculated.

The various groups of animals are listed below in Table 17.

TABLE 17

| Group | | Inhibitor | # mice |
|---|---|---|---|
| 1 | control | | 9 |
| 2 | Docetaxel | Docetaxel 2.5 mg/kg i.v., (qod × 3) | 9 |
| 3 | 10 mg/kg | 10 mg/kg/mouse I.P. daily | 9 |
| 4 | 20 mg/kg | 20 mg/kg/mouse I.P. every 2 days | 9 |

The quantitative results of the experiment are presented in Table 19. For each group, the table reports the caliper measurements and the actual tumor weights obtained as described above. In addition, the table reports the necrotic score, calculated based on the following index shown in Table 18.

TABLE 18

| | | |
|---|---|---|
| N0 | | No visible necrosis |
| N1 | Precursor | Reddened or inflamed; intact tumor |
| N2 | Mild | <10% Tumor necrosis |
| N3 | Moderate | <50% Tumor necrosis |
| N4 | Severe | >50% Tumor necrosis |

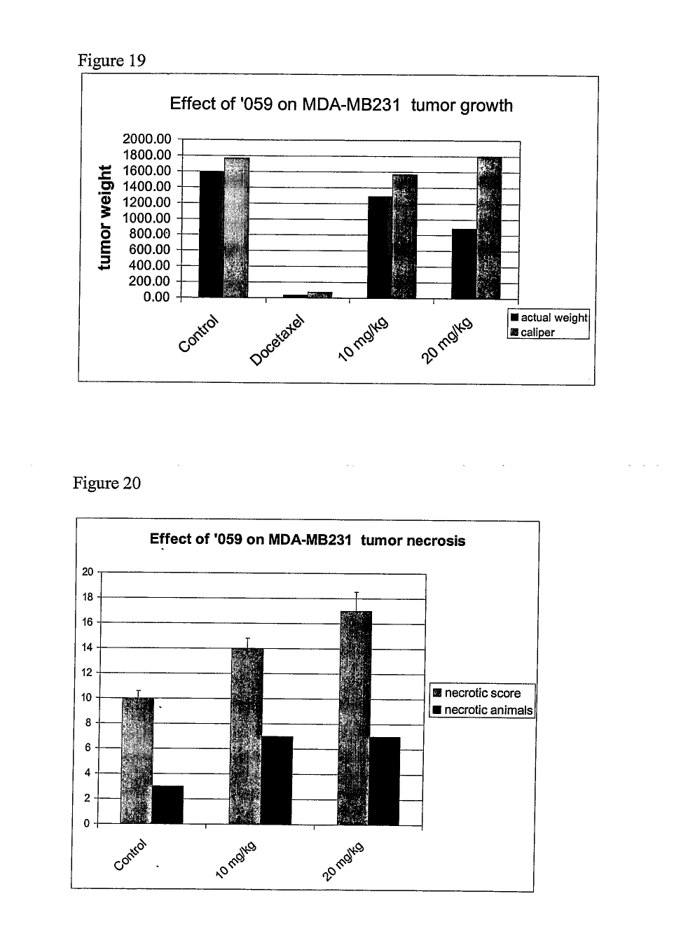

The two sets of data are summarized by the graph in FIG. 19 for the tumor weights and in FIG. 20 for the necrotic scores and the numbers of animals with necrosis. It can be clearly observed that the treated tumors have a large difference in the caliper estimated weight vs. the actual measured weight. Also, the medium actual weight in the treated tumors is smaller than in the controls in a dose dependent fashion with 20 mg/kg<10 mg/kg<controls. Finally both the number of animals with necrosis and the overall level of necrosis are higher in the treated animals in a dose dependent fashion.

The reason for the difference in weight is due to the much larger amount of necrosis present in the treated tumors, both as measured by the necrotic score and by the number of animal with necrosis. Induction of tumor necrosis by antiangiogenic agents is well characterized in the literature and it is part of their antitumor mechanism of action. We conclude therefore that ST100,059 can inhibit the growth and angiogenesis of an human breast cancer tumor grown in immunocompromised mice.

TABLE 19

| | Vehicle | | Docetaxel | | 10 mg/kg 059 | | 20 mg/kg 059 | |
|---|---|---|---|---|---|---|---|---|
| Mouse# | caliper | actual | caliper | actual | caliper | actual | caliper | actual |
| 1 | 1764 | 1482 | 75 | 35 | 1183 | 674 | 1267.5 | 819 |
| 2 | 1470 | 928 | 288 | 225 | 3402 | 2726 | 786.5 | 657 |
| 3 | 2176 | 2082 | 75 | 25 | 3240 | 1731 | 1800 | 884 |
| 4 | 1568 | 1596 | 126 | 82 | 2745.5 | 2327 | 405 | 138 |
| 5 | 2250 | 1751 | 126 | 46 | 1568 | 1287 | 2601 | 1400 |
| 6 | 968 | 887 | 87.5 | 54 | 1568 | 831 | 2456.5 | 1368 |
| 7 | 1913 | 1669 | 32 | 10 | 786.5 | 503 | 364.5 | 313 |
| 8 | 2304 | 2373 | 32 | 18 | 320 | 263 | 2432 | 1717 |
| 9 | 1268 | 939 | 48 | 26 | 3610 | 2915 | 2250 | 2039 |
| Median | 1764 | 1596 | 75 | 35 | 1568 | 1287 | 1800 | 884 |
| Average: | 1742 | 1523 | 1742.22 | 1523 | 2047 | 1473 | 1595.889 | 1037.222 |
| SEM: | 154.6 | 175.391 | 154.574 | 175.391 | 407.685 | 331.9851 | 303.1545 | 212.637 |
| Ttest | | | | | | 0.895722 | | 0.097096 |

necrotic score

| | | 10 mg/kg | 20 mg/kg |
|---|---|---|---|
| Mouse# | Vehicle | 059 | 059 |
| 1 | N-0 | N-2 | N-1 |
| 2 | N-4 | N-1 | N-1 |
| 3 | N-0 | N-3 | N-4 |
| 4 | N-0 | N-2 | N-4 |
| 5 | N-0 | N-0 | N-2 |
| 6 | N-0 | N-2 | N-4 |
| 7 | N-0 | N-0 | N-0 |
| 8 | N-3 | N-1 | N-0 |
| 9 | N-3 | N-2 | N-1 |

### Example 11

### ST100,059 Inhibition of VEGF Induced Intracellular Signaling

The anti-angiogenic activities of the peptides were tested by measuring the level of inhibition of VEGF induced intracellular signaling in human umbilical vein endothelial cells (HUVEC), a standard cell line used to test anti-angiogenic compounds. VEGF stimulation of KDR in endothelial cells results in the phosphorylation of MAPK that is detected with antibodies specific for phosphorylated MAPK and not total MAPK.

Cells were maintained in Cambrex EGM-2MV medium. On day one, cells were starved overnight in 1% FBS in M200 medium (Cascade Biologicals). Afterwards, the medium was replaced with serum free medium±peptides and incubated for 2 hours.

Medium was then replaced with serum free medium containing 25 ng/ml of human VEGF165 and incubated for 10 minutes. Cells were then washed with sodium orthovanadate 2 mM in PBS, harvested in NP40 lysis buffer with sodium orthovanadate 2 mM and PMSF 1 mM and then analyzed by Western blot.

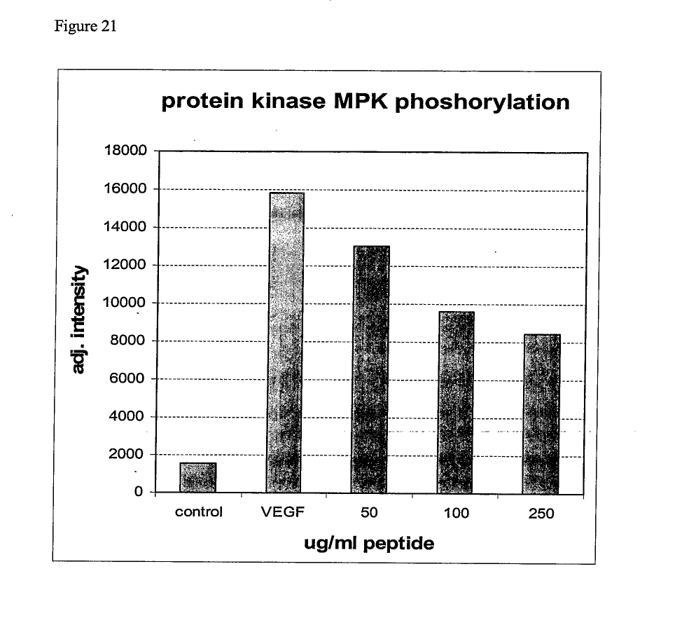

The graph of FIG. 21 shows how increasing concentrations of ST100,059 reduced the level of MPK phoshorylation, as expected for a compound that blocks VEGF binding and therefore activation of its receptor, KDR.

### Example 12

### ST100,059 Inhibition of VEGF Induced Gene Expression Changes

The anti-angiogenic activities of the peptides were tested by measuring the level of inhibition of VEGF induced gene expression changes in HUVEC. VEGF stimulation of the KDR in endothelial cells results in substantial changes in gene expression that has been previously characterized (see patent application 20020132978 Gerber et al.).

Cells were maintained in M200 media (Cascade Biologicals). On day one, cells were starved overnight in 1% FBS in M200 medium (Cascade Biologicals). The morning after the medium was replaced with serum free medium (control) or medium containing 25 ng/ml of human VEGF165 +/−200 microgram/ml of ST100,059 peptide and incubated for 24 hours.

Medium was then aspirated and cells were lysed with Trizol (Gibco) and processed to produce total RNA as described by the manufacturer.

A 10 micrograms aliquot of total RNA was then processed and hybridized on Affymetrix Human U133 Plus 2.0 arrays as described by the manufacturer. The resulting data were analyzed using the Affymetrix GCOS software. It employs statistical algorithms to calculate a quantitative value (Signal Intensity) and a qualitative value (Present or Absent) for each transcript on the array. The data from the 3 samples was then compared to identify those genes that are modulated, either upregulated or downregulated, by VEGF compared to control and whose levels are then brought back to levels similar to control after treatment with ST100,059.

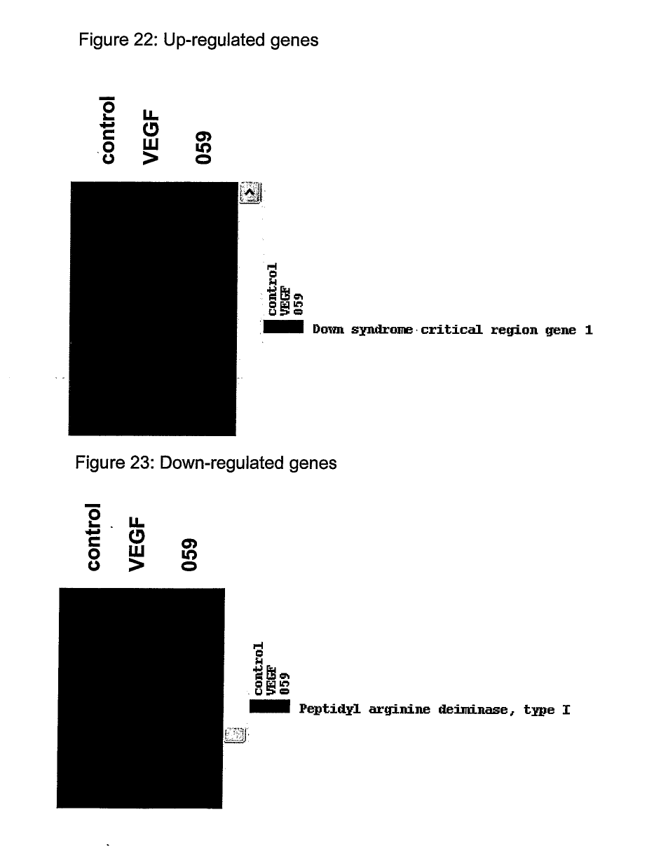

FIGS. 22 and 23 are graphical representations of the results. These results show that ST100,059 is able to inhibit VEGF induced gene expression changes for many genes previously described in the literature. Of interest are those genes described in Yang et al. which are specifically up regulated by the KDR selective mutant of VEGF considering that 059 only blocks VEGF binding to KDR and not FLT-1, the other receptor. OF those genes, several are also upregulated in our experiment and then completely inhibited by 059 including: hydroxysteroid (17-beta) dehydrogenase up 7× Stanniocalcin 1 up 2.4×, Insulin-like growth factor binding protein 5 up 4.5×, gamma synuclein up 2× and ets2 up 2.5 times. The gene Down Syndrome critical region gene 1 and the gene peptidyl arginine deiminase, type 1, are used as examples.

All publications, patents and patent applications discussed herein are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

What is claimed:

1. An anti-angiogenic peptide comprising the amino acid sequence LPPHSS or conservative substitutions thereof.

2. The peptide of claim 1 comprising the amino acid sequence SLPPHSS or conservative substitutions thereof.

3. The peptide of claim 1 comprising the amino acid sequence LPPHSSQ or conservative substitutions thereof.

4. The peptide of claim 1 comprising the amino acid sequence SLPPHSSQ or conservative substitutions thereof.

5. The peptide of claim 1 comprising the amino acid sequence TSLPPHSS or conservative substitutions thereof.

6. The peptide of claim 1 comprising the amino acid sequence LPPHSSQS or conservative substitutions thereof.

7. The peptide of claim 1 comprising the amino acid sequence TSLPPHSSQ or conservative substitutions thereof.

8. The peptide of claim 1 comprising the amino acid sequence SLPPHSSQS or conservative substitutions thereof.

9. The peptide of claim 1 comprising the amino acid sequence TSLPPHSSQS or conservative substitutions thereof.

10. The peptide of claim 1 comprising the amino acid sequence ATSLPPHSS or conservative substitutions thereof.

11. The peptide of claim 1 comprising the amino acid sequence ATSLPPHSSQ or conservative substitutions thereof.

12. The peptide of claim 1 comprising the amino acid sequence ATSLPPHSSQS or conservative substitutions thereof.

13. The peptide of claim 1 comprising the amino acid sequence LPPHSSQSP or conservative substitutions thereof.

14. The peptide of claim 1 comprising the amino acid sequence SLPPHSSQSP or conservative substitutions thereof.

15. The peptide of claim 1 comprising the amino acid sequence TSLPPHSSQSP or conservative substitutions thereof.

16. The peptide of claim 1 comprising the amino acid sequence ATSLPPHSSQSP or conservative substitutions thereof.

17. An isolated peptide comprising the amino acid sequence SLPPHSSQ.

18. An isolated peptide comprising the amino acid sequence TSLPPHSS.

19. An isolated peptide comprising the amino acid sequence LPPHSSQS.

20. The peptide of claim 17 comprising the amino acid sequence TSLPPHSSQ.

21. The peptide of claim 19 comprising the amino acid sequence SLPPHSSQS.

22. The peptide of claim 21 comprising the amino acid sequence TSLPPHSSQS.

23. The peptide of claim 18 comprising the amino acid sequence ATSLPPHSS.

24. The peptide of claim 23 comprising the amino acid sequence ATSLPPHSSQ.

25. The peptide of claim 24 comprising the amino acid sequence ATSLPPHSSQS.

26. The peptide of claim 19 comprising the amino acid sequence LPPHSSQSP.

27. The peptide of claim 26 comprising the amino acid sequence SLPPHSSQSP.

28. The peptide of claim 27 comprising the amino acid sequence TSLPPHSSQSP.

29. The peptide of claim 28 comprising the amino acid sequence ATSLPPHSSQSP.

30. The peptide of claim 23 comprising the amino acid sequence ATSLPPHSSLQT.

31. The peptide of claim 29 comprising the amino acid sequence ATSLPPHSSQSPL.

32. The peptide of claim 29 comprising the amino acid sequence ATSLPPHSSQSPRAL.

33. An isolated peptide comprising the amino acid sequence SLPPRALQ.

34. An isolated peptide comprising the amino acid sequence TSLPPRAL.

35. An isolated peptide comprising the amino acid sequence LPPRALQS.

36. The peptide of claim 30 comprising the amino acid sequence TSLPPRALQ.

37. The peptide of claim 32 comprising the amino acid sequence SLPPRALQS.

38. The peptide of claim 34 comprising the amino acid sequence TSLPPRALQS.

39. The peptide of claim 31 comprising the amino acid sequence ATSLPPRAL.

40. The peptide of claim 36 comprising the amino acid sequence ATSLPPRALQ.

41. The peptide of claim 37 comprising the amino acid sequence ATSLPPRALQS.

42. The peptide of claim 32 comprising the amino acid sequence LPPRALQSP.

43. The peptide of claim 39 comprising the amino acid sequence SLPPRALQSP.

44. The peptide of claim 39 comprising the amino acid sequence TSLPPRALQSP.

45. The peptide of claim 41 comprising the amino acid sequence ATSLPPRALQSP.

46. An isolated peptide comprising the amino acid sequence WLPPHSS.

47. The peptide of claim 43 comprising the amino acid sequence ATWLPPHSSQSP.

48. An isolated peptide comprising the amino acid sequence WLPPRAL.

49. The peptide of claim 45 comprising the amino acid sequence ATWLPPRALQSP.

50. The peptide of any of claims 1-49, wherein said peptide comprises L-amino acids.

51. The peptide of any of claims 1-49, wherein said peptide comprises D-amino acids.

52. The peptide of any of claims 1-49 where one or more peptide bonds are reduced.

53. A retro inverso peptide comprising the reverse amino acid sequence of the peptide of claim 51.

54. An isolated peptide comprising the amino acid sequence PSQSSHPPLSTA.

55. The peptide of any of claims 1-49 or 54, wherein said peptide comprises an acetylated amino terminus.

56. The peptide of any of claims 1-49 or 54, wherein said peptide comprises an amidated carboxy terminal.

57. The peptide of any of claims 1-49 or 54, wherein said peptide is conjugated to a moiety that enhances serum stability.

58. The peptide of claim 57, wherein said moiety is selected from the group consisting of albumin, immunoglobulins and fragments thereof, transferrin, lipoproteins, liposomes, α-2-macroglobulin and α-1-glycoprotein, polyethelene glycol and dextran.

59. A pharmaceutical composition comprising the peptide of any of claims 1-49 or 54.

60. The composition of claim 59 further comprising a pharmaceutically acceptable carrier.

61. The composition of claim 60, wherein said carrier is a liposome forming lipid.

62. The method of claim 60, wherein the composition is administered in a liposome delivery vehicle.

63. The composition of claim 59, further comprising a polymeric carrier that permits controlled release of said peptide, said polymeric carrier being selected from the group consisting of controlled release nanoparticle and microparticle.

64. The composition of claim 63, wherein said microparticle is a microbead or a biodegradable microsphere.

65. The composition of claim 64, wherein said biodegradable microsphere comprises a poly(lactic acid-co-glycolic acid) (PLGA) copolymer.

66. The composition of claim 60, wherein the composition is formulated for aerosol delivery.

67. The composition of claim 60, wherein the composition is formulated as a nasal spray.

68. The composition of claim 60, wherein the composition is formulated for oral administration.

69. The composition of claim 60, wherein the composition is formulated as a tablet, pill or capsule.

70. The composition of claim 60, wherein the composition is formulated as a depot or suppository.

71. The composition of claim 59 further comprising one or more additional anti-angiogenic or anticancer compounds.

72. A method for reducing vascular endothelial growth factor (VEGF)-mediated angiogenesis, comprising contacting a cell expressing kinase domain receptor (KDR) with the peptide of any of claims 1-49 or 54 such that VEGF-mediated angiogenesis is reduced.

73. A method for blocking VEGF binding to a KDR or a KDR peptide, comprising contacting said KDR or said KDR peptide with the peptide of any of claims 1-49 or 54 such that VEGF binding is blocked.

74. The method of claim 73, wherein said KDR or KDR peptide is expressed on the surface of a cell.

75. The method of claim 74, wherein said cell is maintained in vitro.

76. The method of claim 74, wherein said cell is selected from the group of prokaryotic and eukaryotic cells.

77. The method of claim 74, wherein said cell is in vivo.

78. The method of claim 74, wherein said cell is in a subject diagnosed with cancer.

79. The method of claim 73, wherein said KDR or KDR peptide is displayed on a surface.

80. The method of claim 74, wherein said KDR or KDR peptide is displayed in a peptide array on a surface.

81. A method of treating a patient diagnosed with cancer with a therapeutically effective amount of the peptide of any of claims 1-49 or 54, comprising administering said peptide to said patient such that spread of said cancer is reduced or inhibited.

82. The method of claim 81, wherein said cancer is a solid tumor cancer selected from the group consisting of kidney, colon, ovarian, prostate, pancreatic, lung, brain, breast and skin.

83. A method of treating a patient diagnosed with a angiogenesis-associated eye disease with a therapeutically effective amount of the peptide of any of claims 1-49 or 54, comprising administering said peptide to said patient such that said eye disease is reduced or inhibited.

84. The method of claim 83, wherein said eye disease is selected from the group consisting of retinopathy of prematurity, diabetic retinopathy, retinal vein occlusion, macular degeneration and neovascularization associated with corneal injury or grafts.

85. A method of treating a patient diagnosed with an angiogenesis-related disease with a therapeutically effective amount of the peptide of any of claims 1-49 or 54, comprising administering said peptide to said patient such that said angiogenesis-related disease is reduced or inhibited.

86. The method of claim 85, wherein said angiogenesis-related disease is selected from the group consisting of hemangiomas, rheumatoid arthritis, atherosclerosis, idiopathic pulmonary fibrosis, vascular restenosis, arteriovenous malformations, meningiomas, neovascular glaucoma, psoriasis, angiofibroma, hemophilic joints, hypertrophic scars, Osler-Weber syndrome, pyogenic granuloma, retrolental fibroplasias, scleroderma, trachoma, vascular adhesion pathologies, synovitis, dermatitis, endometriosis, pterygium, wounds, sores, and ulcers (skin, gastric and duodenal).

87. The method of claim 73, wherein said KDR is contacted with said peptide in the presence of VEGF.

88. The method of claim 73, wherein said KDR is contacted with said peptide prior to being exposed to VEGF.
